(19) United States
(12) Patent Application Publication
(10) Pub. No.: US 2026/0357322 A1
(43) Pub. Date: Nov. 12, 2026

(54) HIGH-THROUGHPUT MAPPING AND NORMALIZATION FOR GENOMIC METHODS

(71) Applicant: The Regents of the University of California, Oakland, CA (US)

(72) Inventors: Yuwei Cao, La Jolla, CA (US); Sven Heinz, San Diego, CA (US); Alon Goren, San Diego, CA (US); Lauren Patel, La Jolla, CA (US); Christopher Benner, Poway, CA (US)

(21) Appl. No.: 19/139,250

(22) PCT Filed: Dec. 14, 2023
(86) PCT No.: PCT/US2023/084134

§ 371 Date: Jun. 13, 2025

Related U.S. Application Data
(60) Provisional application No. 63/432,625, filed on Dec. 14, 2022.

(51) Int. Cl. C12Q 1/6804; G16B 35/10; G16B 35/20

(52) U.S. Cl. CPC C12Q 1/6804; G16B 35/10; G16B 35/20

(57) ABSTRACT

Compositions and methods are described herein that are useful for improving the normalization of next-generation sequencing data, such as chromatin immunoprecipitation sequence (ChIP-seq) data. The compositions and methods include use of chromatin from two or more unrelated species as a background that is expected to be invariant. The use of chromatin from two or more unrelated species as described, herein improves the resolution of various perturbation conditions on the target species more accurately, in particular given a specific ratio between the two species.

### CROSS-REFERENCE TO RELATED APPLICATION

This application claims the priority of U.S. provisional application Ser. No. 63/432,625, filed Dec. 14, 2022, the disclosure of which is incorporated herein by reference in its entirety as if fully set forth herein.

### STATEMENT OF GOVERNMENT SUPPORT

This invention was made with government support under grant number SPMCB-200358 awarded by the National Science Foundation, R01MH127077 awarded by the National Institutes of Health, grant number 2019688 awarded by the Binational Science Foundation (a joint program with the National Science Foundation), and grant number 2003358 awarded by the National Science Foundation, Division of Molecular and Cellular Bioscience NSF/MCB-BSF. The government has certain rights in the invention.

### BACKGROUND

Chromatin immunoprecipitation followed by sequencing (ChIP-seq) is a method for studying the genomic localization of DNA-associated proteins. ChIP-seq is widely used to study histone modifications, chromatin regulators, transcription factors, and other DNA-associated proteins. The overall experimental workflow of ChIP-seq involves multiple steps that increase the risk of introducing inconsistency within experiments and between groups. These challenges can be partially addressed by the incorporation of automated liquid handling platforms to improve the robustness of the process. Yet, most of the previous efforts have automated only a subset of the ChIP-seq steps. Additionally, the majority of the ChIP-seq protocols—both automated and manual—are limited in their ability to efficiently map non-histone DNA-associated proteins, such as chromatin regulators that may indirectly interact with the genome (e.g., by being recruited to modified histones or histone variants). Further, ChIP-seq is “semi-quantitative”, i.e. each locus can be assigned a score such as number of reads. While approaches for normalization of between samples in epigenomic studies using “spike-in” normalization controls were introduced about a decade ago, the implementation of such methods suffers from high variability, and this lack of standardization and rigor has led to discordant conclusions. Therefore, a need exists for agents, procedures, and methods that can improve normalization of ChIP-seq data.

### SUMMARY

Compositions and methods are described herein that are useful for improving the normalization of next-generation sequencing data, such as chromatin immunoprecipitation sequence (ChIP-seq) data. The compositions and methods include use of chromatin from two or more unrelated species as a background that is expected to be invariant. The use of chromatin from two or more unrelated species as described herein improves the resolution of various perturbation conditions on the target species more accurately, in particular given a specific ratio between the two species.

Any evaluation of global changes in histone modifications and similar epigenetic markers measured with sequencing data includes quantitative analysis. Methods are provided herein for various normalization strategies to monitor variations such as pipetting inconsistency or other noises in spike-in preparation. A novel approach is provided herein that overcomes such issues by using two or more spike-ins and thus allows monitoring of any changes in the ratio between the spike-ins that may originate from sample preparation.

A linear relationship between the ratio of chromatin added and the ratio of reads obtained for each species is achieved. Using chromatin from other species allows identification of anomalies in data and separate biologically interesting differences from technical variation.

In one embodiment, the disclosure provides a method for normalizing chromatin immunoprecipitation (ChIP) sequence data comprising: obtaining a culture of animal cells; arresting a cell cycle of the animal cells; crosslinking the animal cells; harvesting the animal cells; sonicating the animal cells to produced sheared chromatin; spiking the sheared chromatin with at least two exogenous chromatin samples from two species that are different from the species of the animal cells to produce a combined chromatin sample, wherein the ratio of the amount of the at least two exogenous chromatin samples is known; immunoprecipitating the combined chromatin sample; producing a ChIP-seq library; amplifying the ChIP-seq library; sequencing the immunoprecipitated combined chromatin sample to produce sequencing data; and normalizing the sequencing data based on the known linear relationship between the at least two exogenous chromatin samples. In one embodiment, the animal cells are arrested in a prometaphase-like state. In one embodiment, the animal cells are crosslinked in Disuccinimidyl glutarate (DSG) with phosphate buffered saline (PBS), formaldehyde, or both. In one embodiment, the ratio of the amount of at least two exogenous chromatin samples is different. In one embodiment, an antibody used in immunoprecipitating the chromatin recognizes an epitope in the chromatin and in the at least two exogenous chromatin samples. In one embodiment, normalization is quantitative. In one embodiment, a liquid handler is used in at least one of the steps of sequencing, immunoprecipitating, producing the ChIP-seq library, amplifying, or normalizing. In one embodiment, the chromatin in the at least two exogenous chromatin samples are sheared.

In one embodiment, the disclosure provides a method of using an automated liquid handler to conduct ChIP-seq reactions comprising: obtaining a culture of animal cells; arresting a cell cycle of the animal cells, crosslinking the animal cells; harvesting the animal cells; sonicating the animal cells to produce sheared chromatin; input the sheared chromatin into a sample plate in an automated liquid handler; actuate the automated liquid handler to at least one of: immunoprecipitate the sheared chromatin; wash and elute the immunoprecipitated sheared chromatin; prepare a library; amplify the library; quantify DNA in the library; sequence the library.

### BRIEF DESCRIPTION OF THE DRAWINGS

The drawings illustrate generally, by way of example, but not by way of limitation, various embodiments discussed herein.

FIG. 1A-1C illustrate the inaccurate application of spike-in normalization. FIG. 1A is a schematic of a proposed regulatory mechanism showing RNAPII directly regulates histone marks from¹¹. FIG. 1B is a graph showing normalized promoter H3K4me3 signal following transcription inhibition with triptolide. The signal appears to reduce vis-à-vis lack of transcription (modified from¹¹). FIG. 1C is a graph showing re-analysis of the data from¹¹: percent of reads aligned to the spike-in genome for ChIP-seq inputs. The y axis is cut due to the range in spike-in. Raw aligned read counts labeled above each bar. Spike-in normalization relies on adding the same amount of exogenous chromatin to each sample, and as shown here, the proportion of reads in the input can be used to QC the amount of spike-in added. Note, the major differences between the conditions reported appear to be highly concordant with the discrepancies between the number of reads aligned to the spike-in genome, thus potentially invalidating the conclusion of this experiment.

FIG. 2 is a graph showing that spike-in reads vary linearly with the amounts of added chromatin. To an equal number of human cells, exogenous chromatin was added, varying both the ratios of spike-in to target and between spike-in species (the targeted ratios are indicated on the x-axis). The ratio of spike-in to human aligned reads in inputs is consistent with the ratio of added chromatin.

FIGS. 3A-3C illustrate how spike-in transcription start site (TSS) signal indicates antibody competition. FIG. 3A is a graph showing the difference in Drosophilia spike-in H3K9ac IP signal from S2 cells, which should always be invariant, in an experiment with constant levels of human H3K9ac. FIG. 3B is a graph showing the difference in Drosophilia spike-in H3K9ac IP signal from S2 cells in an experiment where the global levels of human H3K9ac vary. FIG. 3C is a graph showing a maximum TSS signal in FIG. 3B plotted as a function of increasing human epitope. The line representing signal with a lack of competition indicated. Single spike-in species shown for simplicity, similar results were observed in both species.

FIG. 4 shows that capped small RNA sequencing (csRNA-seq) captures transcriptional initiation in mitotic cells. Mitotic HeLa-S3 were treated with dimethyl sulfoxide (DMSO) or histone-deacetylase 3 (HDAC3) with the HDAC3 inhibitor RGFP966 (CAS No.: 1357389-11-7). University of California, Santa Cruz, (UCSC) browser transcription initiation sites compared with H3K9ac ChIP-seq peaks. Arrows: a csRNA-seq peak that is induced along with the increase in H3K9ac following HDAC3 inhibition.

FIGS. 5A-5B illustrates normalizing to dual spike-in chromatin reveals global changes. Mitotic (low K9ac) and Interphase (high K9ac) cells were mixed in various ratios. H3K9ac around top 500 peaks, read depth normalized (FIG. 5A) or following dual spike-in normalization (FIG. 5B). Inserts: Maximum signal of each sample (peak height) compared to expected value assuming a linear relationship between the proportion of interphase cells and epitope signal, linear model fit.

FIGS. 6A-6C. Mapping HAT chromatin occupancy with automated ChIP-seq. FIG. 6A shows IGV Browser images of HAT1 and EP300. FIG. 6B, FIG. 6C. Metagene plots of HAT1 and EP300 after optimizing automated ChIP-seq conditions. Automated ChIP-seq carried out with BRAVO robotic liquid handling system (hereinafter “Bravo”).

FIGS. 7A-7C. Effect of HDAC inhibitors on mitotic H3K9ac in HeLa-S3 cells. FIG. 7A. Histogram of spike-in H3K9ac around promoters pre- and post-normalization FIG. 7B. UCSC Brower images of inhibitor and non-treated mitotic H3K9ac FIG. 7C. Metagene plots of H3K9ac around promoters, enhancers, and insulators. Inhibitors. CAY10683 targets HDAC2, EX-527 targets SIRTI, MS275 targets HDACs 2 and 3, NT (non-treated synchronized cells), RGFP966 targets HDAC3, Unsync (unsynchronized cells). CAY10683, EX527, MS275, and Unsync ChIP-seq done by the Simon lab at Hebrew University in Jerusalem.

FIGS. 8A-8D. Normalization to spike-in TSS allows observation of HDAC inhibitor effects. FIG. 8A: Histogram of H3K9ac at spike-in genome (galGal6) promoters, read depth normalized. FIG. 8B: Histogram of H3K9ac at spike-in genome promoters normalized to peak TSS signal. FIG. 8C: Histogram of mitotic H3K9ac, read depth normalized. FIG. 8D: Histogram of mitotic H3K9ac, normalized to spike-in peak TSS signal in FIG. 3B. Each treatment has four biological replicates.

FIGS. 9A-9D. Spike-in species exhibit consistent behavior in various shearing and perturbation conditions. FIG. 9A and FIG. 9B show H3K9ac around D. melaniogaster TSS regions, where chromatin was sheared with two different devices, either the Covaris E220 (FIG. 9A) or PIXUL (FIG. 9B). FIG. 9C and FIG. 9D show H3K9ac around G. gallus TSS regions, where chromatin was sheared with Covaris E220 (FIG. 9C) or PIXUL (FIG. 9D). All spike-in chromatin was mixed with HeLa-S3 cells exposed to various perturbation conditions.

FIG. 10. Schematic of an example dual spike-in ChIP-seq method. Hela-S3 cells are synchronized with thymidine and S-Trityl-Cysteine resulting in a prometaphase-like state, then fixed with formaldehyde and harvested. Simultaneously, D. melanogaster (Spike-in 1) S2 cells and G. gallus (Spike-in 2) DT-40 cells are fixed, harvested, and added to the HeLa-S3 cells in predefined ratios. In embodiments, the dual spike-in ChIP-seq method can use species other than D. melanogaster and G. gallus. In embodiments, the dual spike-in ChIP-seq method can use cells from the same species where two or more pre-barcoded chromatin is added at predefined ratios (See Example 4 herein). All subsequent experimental steps are done to the combined sample. Figure created with Biorender.

FIGS. 11A-11B. Spike-in chromatin reads vary linearly with chromatin added. Reads aligning to spike-in genomes vary linearly with chromatin added. FIG. 11A: Cells from each species per condition, each with two biological replicates. FIG. 11B: Calculated ratio of Fly:Human and Chicken:Human aligned reads in H3K9ac ChIP-seq samples and inputs. Reads aligned to a concatenated genome containing all 3 species.

FIG. 12. Spike-in genome quality affects quantitative normalization. The quality of the spike-in genome assembly has a significant impact on the feasibility of quantitative normalization. G. gallus chromatin was initially selected as a spike-in chromatin donor for its relatively close phylogenic distance to mammalian species, since the antibody must recognize the epitope of interest in all species. However, there are also disadvantages to using a less common species. We observed more low-quality aligned reads than expected for the G. gallus genome (lower dots), especially in the input libraries that impacted our normalization consistency.

FIGS. 13A-13B. Improving cost effectiveness with S. cerevisiae chromatin. ChIP-seq of yeast H3K9ac around TSS. H3K9ac antibodies recognize the conserved histone modification H3K9ac in D. melanogaster, G. gallus, and S. cerevisiae. S. cerevisiae was recently substituted for G. gallus to improve cost effectiveness and feasibility of the method. FIG. 13A. Metagene plot of H3K9ac around S. cerevisiae promoters. FIG. 13B. Correlation between human:yeast cell ratio and human:yeast aligned reads from WCE chromatin.

FIG. 14. During mitosis a nucleosome with unique epigenetic status enters the NDR. Previous data collected by the Simon lab at Hebrew University of Jerusalem demonstrated that a nucleosome enters the NDR region of some promoters in mitosis. Here promoters are classified according to their NDR occupancy and the epigenetic status of the NDR nucleosome. Group 1 promoters retain their nucleosome-depleted region (NDR). Group 2 promoters lose their NDR in mitosis, and the nucleosome entering the region is methylated but not acetylated. Group 3 promoters also lose their NDR in mitosis, and the nucleosome entering the region is both methylated and acetylated.

FIGS. 15A-15D. The mitotic NDR nucleosome becomes acetylated after HDAC inhibition. FIG. 15A and FIG. 15B. Histogram of H3K4me3 in mitotic cells around Group 1 and Group 2 promoters defined in FIG. 11. H3K4me3 occupancy is not affected by various HDAC2 (CAY10683) or HDAC3 (RGFP966) inhibition. Each inhibitor treatment has two biological replicates. FIG. 15C and FIG. 15D. Histogram of Group 1 and Group 2 Promoters defined in 7, with HDAC3 inhibition (RGFP Treatment) in blue. The shaded confidence interval represents 2 standard deviations from the mean of 4 biological replicates. Bin size of 25 bp for all histograms.

FIG. 16 Overview of the ChIP-seq workflow with the automated steps highlighted in gray boxes and evaluated conditions in yellow boxes.

FIGS. 17A-17P show the results of various methods of ChIP-seq. FIG. 17A is an image of 2% Agarose Gel for PIXUL vs Covaris E220 Shearing of FA-crosslinked HeLa-S3 cells. 60-minute shearing of single crosslinked HeLa-S3 cells on PIXUL is shown to be consistent. PIXUL is as consistent as Covaris E220 and allows quicker sheering of multiple samples. FIG. 17B are scatterplots showing that automated and manual ChIP-seq have a similar signal-to-noise (SNR) ratio under the indicated buffer and crosslinker conditions for the histone modifications H3K27ac and H3K27me3. The X-axis shows the read counts (log 2) using the automated ChIP-seq method and the Y-axis shows the read counts (log 2) for a ChIP-seq prepared manually. FIG. 17C shows IGV Genome Browser tracks of read coverage for H3K27ac and H3K27me3 ChIP-seq. FIG. 17D are heatmaps of all genes around the TSS for H3K27ac or the peaks for H3K27me3 showing that automated and manual ChIP-seq have a similar SNR for the histone modifications H3K27ac and H3K27me3. FIG. 17E is a 2% Agarose Gel for PIXUL shearing time (min) compared with DSG/FA double crosslinked HeLa-S3 cells. The gel shows increased shearing process time for DSG/FA+LB3 conditions as compared to for DSG/FA+EMPIGEN® BB conditions. EMPIGEN® BB is a 30% aqueous solution of the detergent lauryl betaine, LB3 is lysis buffer 3, DSG is dimethyl pimelimidate (crosslinker), and FA is formaldehyde (crosslinker). FIG. 17F shows IGV Genome Browser tracks of read coverage for H3K27ac ChIP-seq with DSG/FA crosslinking or FA crosslinking. FIG. 17G are heatmaps of all genes around a transcription stop site (TSS). FIG. 17H is a metagene plot of average tag density around the TSS for H3K27ac ChIP-seq. FIG. 17I are IGV Genome Browser tracks of read coverage, FIG. 17J are heatmaps of all genes around the TSS for H3K27me3 ChIP-seq with DSG/FA crosslinking or FA crosslinking. FIG. 17F-17J show that single crosslinking with FA and double crosslinking with DSG+FA have similar ChIP-seq SNR for H3K27ac and h3K27me. FIG. 17K are IGV Genome Browser tracks of read coverage of automated vs manual ChIP-seq with DSG/FA crosslinking or FA crosslinking. FIG. 17L is a metagene plot of average tag density along the gee bodies for SETD5 ChIP-seq, showing that double crosslinking with DSG+FA improves SNR for the chromatin regulator SETD5. FIG. 17M shows IGV Genome Browser tracks of read coverage of automated vs manual ChIP-seq. FIG. 17N is a metagene plot of average tag density around the TSS for H3K27ac ChIP-seq. FIG. 17O are IGV Genome Browser tracks of read coverage and FIG. 17P, are heatmaps of all genes around the TSS for H3K27me3 ChIP-seq. FIG. 17M-17P show that compared to LB3, the lysis buffer Empigen BB reduces the SNR in H3K27ac and H3K27me3 ChIP-seq. All aforementioned ChIP-seq experiments are done in HeLa-S3 cells.

FIGS. 18A-18D shows results of ChIP-seq from samples immunoprecipitated with SETD5 Polyclonal Antibody. FIG. 18A shows IGV Genome Browser tracks of read coverage for DNA bound by SETD5, as immunoprecipitated with SETD5 Polyclonal Antibody, under DSG/FA+LB3 conditions as compared to for DSG/FA+EMPIGEN® BB conditions. FIG. 18B is a metagene plot of average tag density along the gene bodies for SETD5 ChIP-seq, showing that SETD5 antibody titration by automated ChIP-seq identifies optimal conditions (both antibody and lysis buffer) for the chromatin regulator SETD5. FIG. 18C are IGV Genome Browser tracks of read coverage and FIG. 18D are heatmaps of all genes around TSS for HAT1 and EP300 ChIP-seq experiments performed with the automated workflow, showing that automated ChIP-seq identifies optimal lysis buffer condition for chromatin regulator HAT1 and EP300. All aforementioned ChIP-seq experiments are done in HeLa-S3 cells.

### DETAILED DESCRIPTION

Transcription and histone acetylation are closely linked processes at the core of gene regulation. Recent studies differ on whether RNA Polymerase II (RNAPII) recruitment and transcription precedes histone acetylation, and thus this mark serves only as a “moonlighter” involved in gene regulation by other mechanisms, or whether histone acetylation provides critical functions such as weakening the association between the DNA and nucleosomes, increasing accessibility, and facilitating transcription initiation/elongation. Regardless of the hypothesis, perturbation of either phenomenon results in global changes in transcription and/or histone acetylation, thus requiring a highly precise and well-calibrated quantitative approach.

Multiple attempts have been made to apply ChIP-seq normalization to quantify epigenetic marks more accurately. ChIP-Rx, which uses orthogonal chromatin from a separate species as a “spike-in” internal control, emerged as one of the earliest and most widely used tools, and has been adapted in many studies. However, a lack of rigorous normalization protocols has led to discordant conclusions in epigenomics research. For example, a high impact study in Nature Genetics reported that many histone methylation/acetylation marks appear to be dependent on transcription, as inhibition of RNAPII results in their depletion one and four hours post-Triptolide treatment. The number of spike-in reads were too low and inconsistent (the ratio of spike-in/target reads varied by up to ~100 fold) to provide adequate genome coverage and accurate quantification. Concerningly, the variation in read ratios mirrored reported normalized results suggesting these results may be an artifact of an inappropriate spike-in application. This is not an isolated issue, many spike-in/other normalization approaches have key limitations (Table1) that significantly affect the obtained results.

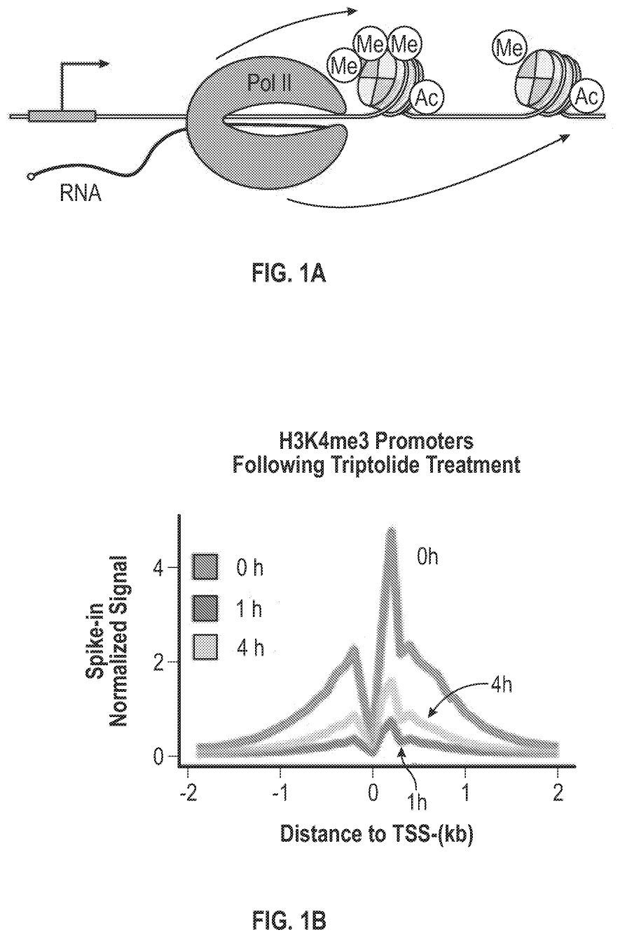

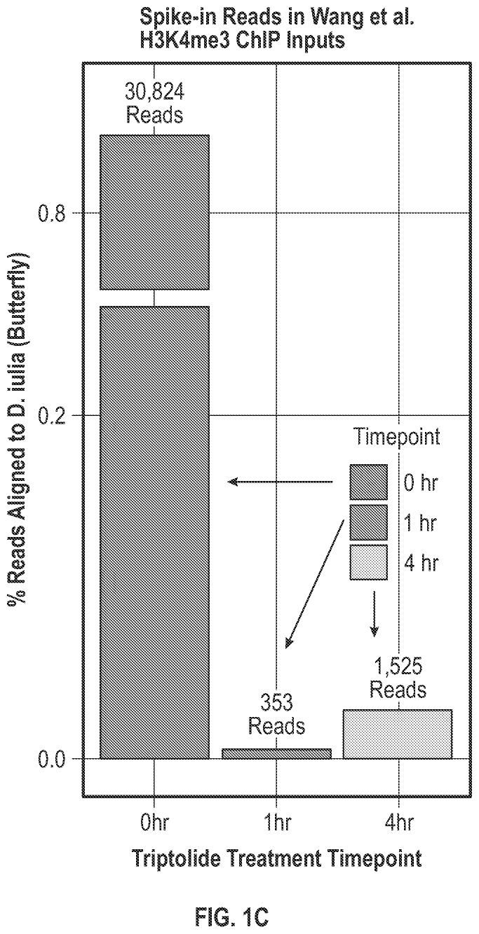

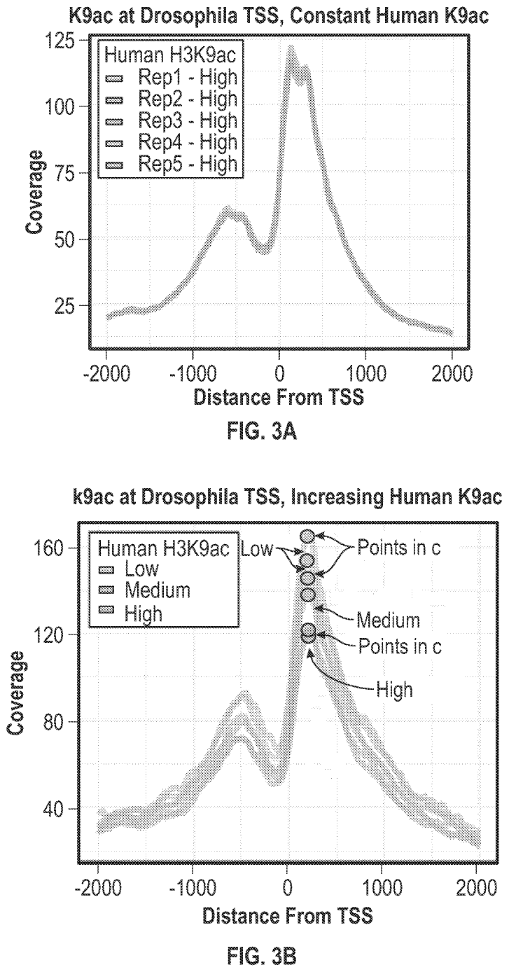

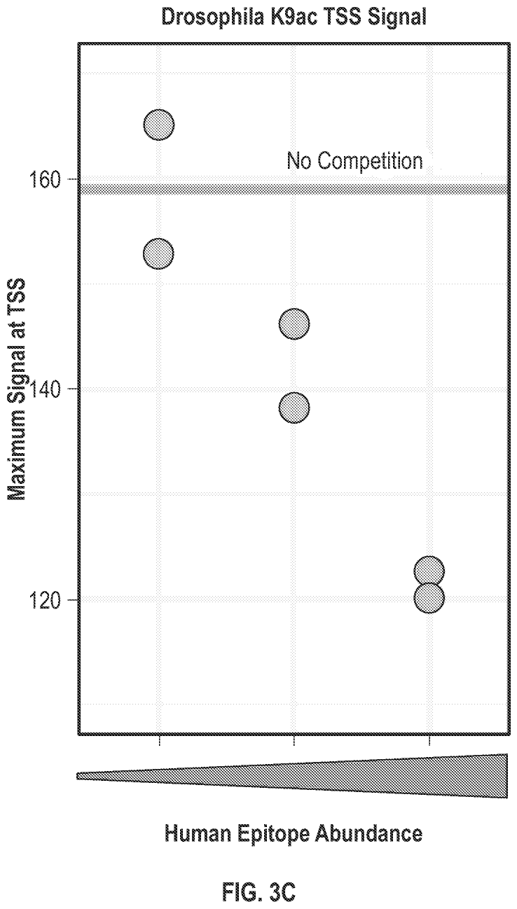

TABLE I

| Normalization tool overview | Key limitations |
|---|---|
| ChIP-Rx: Common antibody for | No QC of the spike-in ratios; no |
| Hs/Dm9 | modeling of antibody competition |
| | (FIG. 3). |
| Hs/Mm with common antibody 10 | Significant overlap between genomes; |
| | no modeling of antibody competition. |
| Epicypher ICeChIP: Synthetic | Limited to study of histone marks |
| nucleosome spike-in18 | only; cannot support evaluation of |
| | shearing or background. |
| Parallel ChIP: Spike-in specific | Significant overlap between genomes, |
| (Dm) and common antibodies | separate antibody increases |
| (Hs) for Mm target species19 | experimental variation. |
| Spike-in specific antibody | Specific antibody may introduce |
| (Dm).20 | experimental error, no modeling of |
| | antibody competition. |
| SNP-ChIP: Use SNPs to enable | Limited in application depending on |
| spike-in of member of the same | target genome. |
| species in S. cerevisiae21 | |
| Use non-transcribed regions in | Assumes background does not change |
| target species as background3 | in response to variations in the |
| | signal, no modeling of antibody |
| | competition. |
| Use CTCF peaks as background, | Spike-in reads vary by ~100 fold, |
| D. iulia (butterfly) spike-in11 | reference genome not well annotated, |
| | background regions not confirmed to |
| | be invariant (FIG. 1). |

Described herein are methods that provide a rigorously benchmarked approach to quantification of sequencing data, identify the necessary tests to verify its accuracy, and apply it to the biological question of how transcription and histone acetylation are interrelated.

### Diverse ChIP-Seq Normalization Strategies Create Uncertainty in Interpretation

ChIP-seq (Chromatin Immunoprecipitation sequencing) has often been a workhorse for measuring histone modifications, transcription factors, and chromatin regulators. Traditional ChIP-seq normalization is done using relative level difference, scaling to the total read depth. Modified versions of this method consider different signal/noise ratios between samples, but these assume that most common peaks should have similar binding intensities among conditions. In general, assumptions for relative level difference do not hold if the genome-wide enrichment levels change drastically; in these cases, adding spike-in chromatin from a separate species can account for experimental variation and reveal large global changes in the target binding. ChIP-Rx is one commonly used approach where a fixed amount of spike-in is added to linearly transform the IP signal by the ratio of spike-in/target reads.

ChIP-Rx has no quality control (QC) metrics to assess unexpected variation in the amount of input material, the impact of antibody competition or other factors affecting enrichment. Table I summarizes the main normalization approaches, highlighting the variability in spike-in species, choice of common or specific antibody, and downstream method of quantification. Lack of a standardized spike-in protocol can lead to inconsistent conclusions, as the different assumptions used can obscure biases unique to each method (FIGS. 1A-1C).

### Contradicting Evidence for the Relationship Between Histone Marks and Transcription.

Many histone marks are highly linked to transcription, which makes it difficult to distinguish causality from correlation. Advances in nascent RNA sequencing tools have gone beyond measuring steady-state mRNAs and can now detect newly transcribed RNAs²²⁻²⁴. These methods offer a unique opportunity to better characterize the relationship between histone marks, such as acetylation, and transcriptional regulation. Current literature suggests multiple theories regarding the relationship between histone acetylation and transcription, including: (i) histone deacetylases (HDACs) help negatively regulate gene expression by inhibiting the release of paused RNAPII, which would implicate histone deacetylation in a critical step of transcriptional regulation²⁵. Perturbation of acetylation impairs transcription dependent on core regulatory transcription factors, implying that acetylation is required for transcription²⁶; (ii) paradoxically there is evidence of the reverse: that transcription itself is a prerequisite for histone acetylase activity³. Predicting histone marks from transcriptional data or studying the effect of transcriptional inhibition is difficult given the interconnection between transcriptional activity and acetylation^{27,28}.

### Mitosis as an Ideal System to Study the Effects of Global Reduction and Buildup of Histone Acetylation and Transcription

Mitosis is a critical phase of the cell cycle that requires careful regulation to ensure cell identity and function are properly relayed to daughter cells^{29,30}. During mitosis, the genome undergoes a significant restructuring resulting in a global reduction of both transcription and histone acetylation³¹⁻³³. Studying the regulatory mechanisms responsible for the reduction in transcription and acetylation is therefore an intriguing way to gain insight about their relationship. The reduction of histone acetylation serves a practical purpose at least in part, as histone H3 lysine 9 (H3K9) deacetylation and subsequent methylation are required for proper chromosome condensation^{34,35}. However, promoters with active acetylation marks appear to have ongoing transcription even in the mitotic stages when chromatin is most compact, as enhancers retaining the mark indicating acetylation of the lysine residue at N-terminal position 27 of the histone H3 protein (H3K27ac) during mitosis are associated with cell type-specific genes and are among the first genes to be reactivated upon mitotic exit³⁶⁻⁴¹, suggesting these acetylated histones may act as “bookmarks” with regulatory significance. The shift in balance of histone deacetylases (HDACs) and histone acetyltransferases (HATs) activity might be critical for suppressing transcription during mitosis and determining the impact of perturbing acetylation in this system can provide an understanding of how acetylation and transcription interact in other major cellular processes.

Use of Spike-In Chromatin or RNA from Two Separate Species in ChIP-Seq

To overcome the limited standardization of current methods (Table1), provided herein are methods for rigorous normalization of next-generation sequencing (NGS)-based data. The method provides multiple validations and quality controls to identify variations with high sensitivity and minimize bias.

The normalization method's use of two spike-ins at constant amounts means that technical variations between replicates will be easily detected as changes to the ratio of reads aligned to each species. The use of spike-ins derived from two separate species can enable identification of the dynamic range for each NGS-based normalization method. For instance, the dynamic ranges may vary between chromatin and RNA-based assays due to differences between the methods in signal/noise ratios and peak distributions. There is a linear relationship between the ratio of chromatin added and the ratio of reads obtained for each species within this range and each assay experiences biases in detection of low or high abundance species.

In conjunction with the above strategies, an automated liquid handler is used perform automated ChIP-seq⁴² to further increase throughput and reproducibility and minimize errors and costs. Each experiment can be conducted with multiple (at minimum 3-4) replicates, thus increasing the statistical power to identify variations in signal between conditions. This quantification approach addresses a fundamental question surrounding epigenetic and transcriptional control.

### Dual Spike-In Normalization Reveals Impact of Spike-In Species on Accuracy of Normalization.

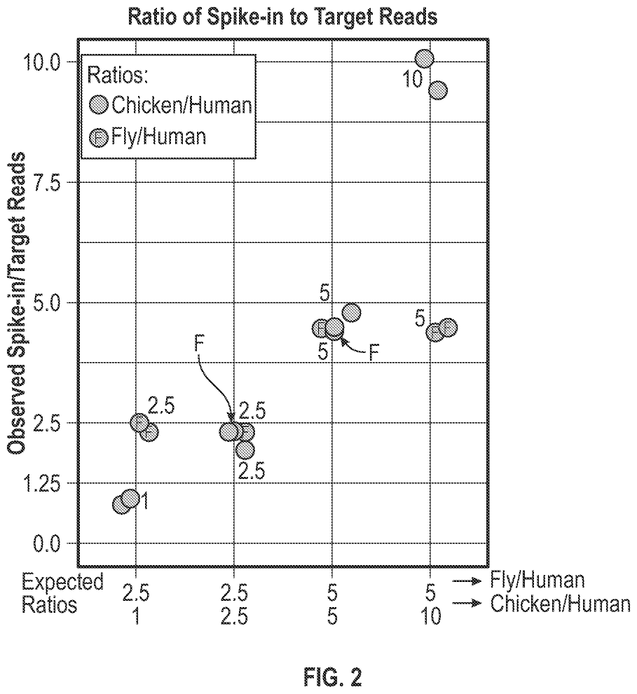

To establish a dual spike-in approach, exogenous chromatin from fly (D. melanogaster S2) and chicken (G. gallus DT-40) cells were used. While using the fly and chicken chromatin, an improved assessment of the accuracy of technical replicates was shown (FIG. 2) and a key limitation of using certain genomes for spike-in normalizations was found. In particular, genomes less widely used such as G. gallus are more likely to have lower quality reads align; the evolutionary proximity to humans also increases the chances of misalignment. This issue was circumvented by aligning to concatenated genomes and filtering appropriately for Bowtie2⁴³ MAPQ, resulting in minimal misattributed reads (<0.025%), but it was observed that other alignment methods and less stringent filtering had significant impacts on the number of reads counted as spike-in aligned (spike-in reads varied by 5-10%). Thus, when choosing an exogenous species for spike-in one should account for the quality of the reference genome.

Following the challenges observed in using the G. gallus reference genome, the chicken cells were replaced with yeast S. cerevisiae cells as a second species spike-in. The use of yeast chromatin provides multiple advantages: (1) the genome is well characterized, (2) there is less overlap between S. cerevisiae and H. sapiens genomes, (3) smaller genome size means fewer reads are needed to obtain sufficient coverage, thus reducing costs, and (4) yeast cells are easier to obtain and maintain. Yet, in other applications focused on non-histone DNA-associated proteins, the larger evolutionary distance between yeast and human may limit the number of conserved epitopes recognizable by a shared antibody. In such cases the chicken spike-in could be used, along with updates to the G. gallus reference genome.

### Spike-In TSS Behavior Indicates Antibody Competition

The impact of antibody competition is an overlooked detail when choosing a ChIP-seq normalization method. Previous data^{29,36,41,44} shows a strong, global decrease of acetylation occurs in mitosis. The large changes in mitotic and interphase histone acetylation is a scenario where read-depth normalization alone is not sufficient. For example, consider the difference in Drosophila spike-in H3K9ac IP signal from S2 cells, which should always be invariant, in an experiment with constant levels of human H3K9ac (FIG. 3A) versus an experiment where the global levels of human H3K9ac vary (FIGS. 3B-3C). As the amount of spike-in is invariant, no change in the signal would be expected (FIG. 3A). Yet, as human H3K9ac increases (FIG. 3B), a loss of spike-in signal is observed at the Drosophila TSS where spike-in epitope is out-competed for antibody. The degree of competition can be estimated by quantifying the relative drop in signal (FIG. 3C). The importance of modeling antibody competition in a computational pipeline is apparent.

Measuring Transcription Initiation with csRNA-Seg to Identify Impact of Increased Acetylation in Mitosis.

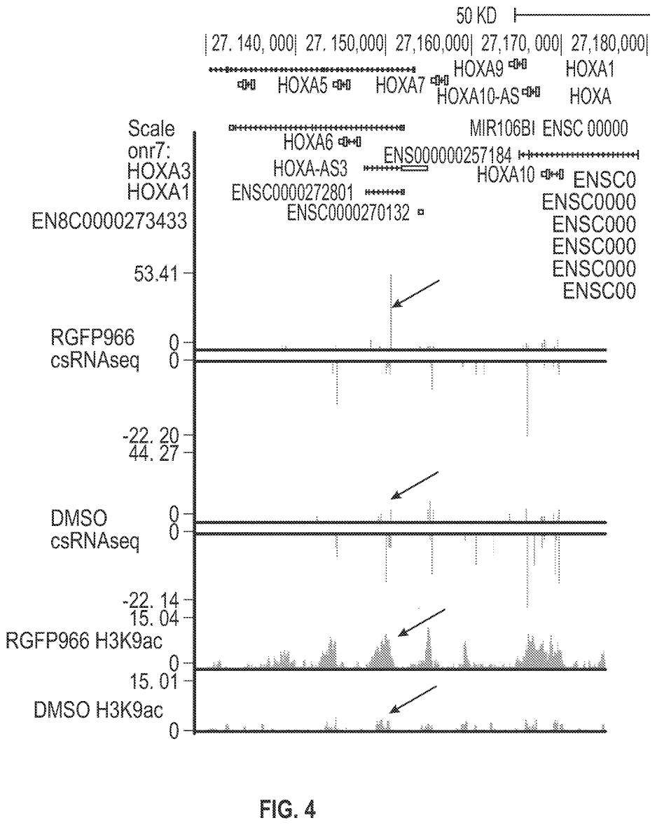

Our lab developed a technique called short capped RNA-seq (csRNA-seq) which takes advantage of the combination of short size (20-70 nt) and 5′ cap that is unique to newly initiated RNAs by RNAPII23. By isolating these RNA species and comparing to a background input of all short RNAs we can identify nascent strand-specific transcription initiation sites and quantify their levels at single nucleotide resolution. This assay can be used to measure time resolved changes in transcription initiation following perturbation of histone acetylation (down to 5 minutes intervals) without the need for nuclei isolation or radioactive labeling in similar methods. FIG. 4 shows the output of csRNA-seq compared to ChIP-seq in mitotic HeLa-S3 cells. To study the involvement of HDAC3 in regulation of H3K9ac during mitosis, we treated mitotic HeLa-S3 cells with the HDAC3 inhibitor RGFP966 and show that acetylated histone levels are elevated (FIG. 4). The arrows show an induced TSS in a region with increased acetylation following HDAC3 inhibition in mitosis. These data support our hypothesis that HDAC3 is the major deacetylase targeting H3K9ac during metaphase, the stage of global minimum histone acetylation44, and in combination with csRNA-seq data provide an assessment of the impacts of increased acetylation on transcription during a cellular process with naturally low acetylation.

### Dual Spike-In Normalization Reveals Global Decrease in Acetylation Observed in Mitotic Cells.

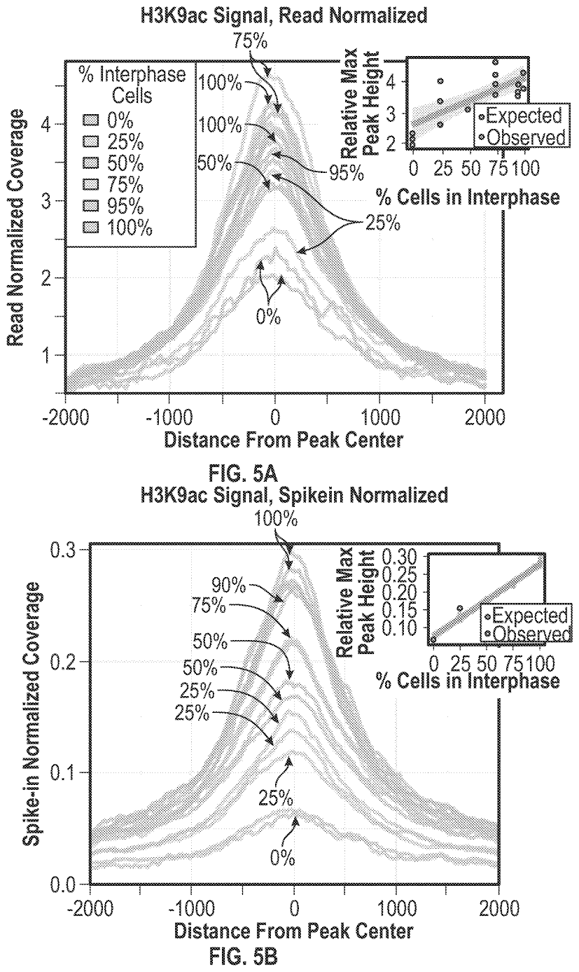

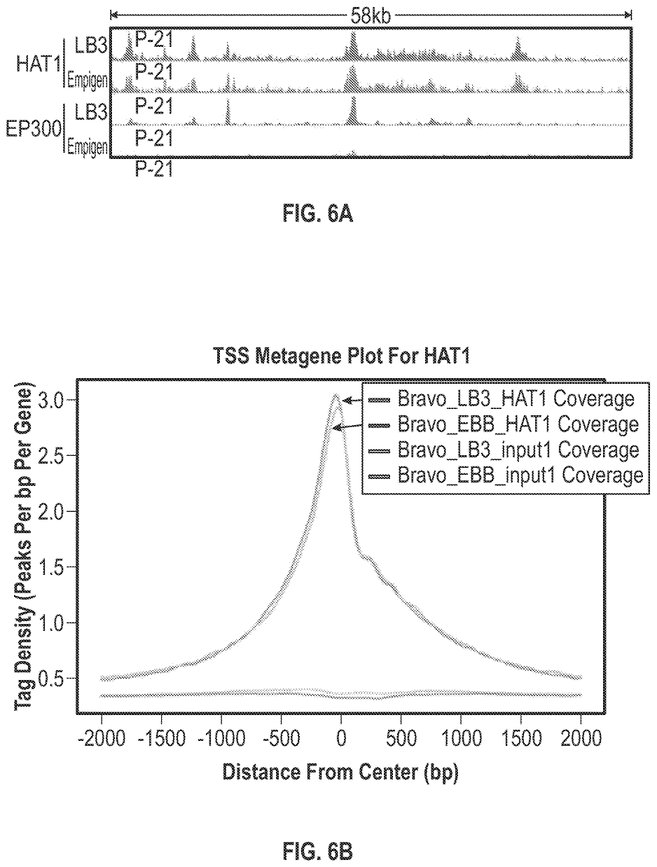

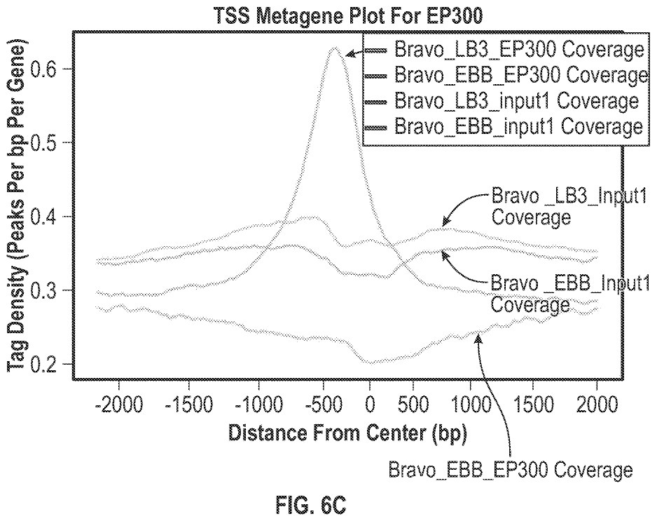

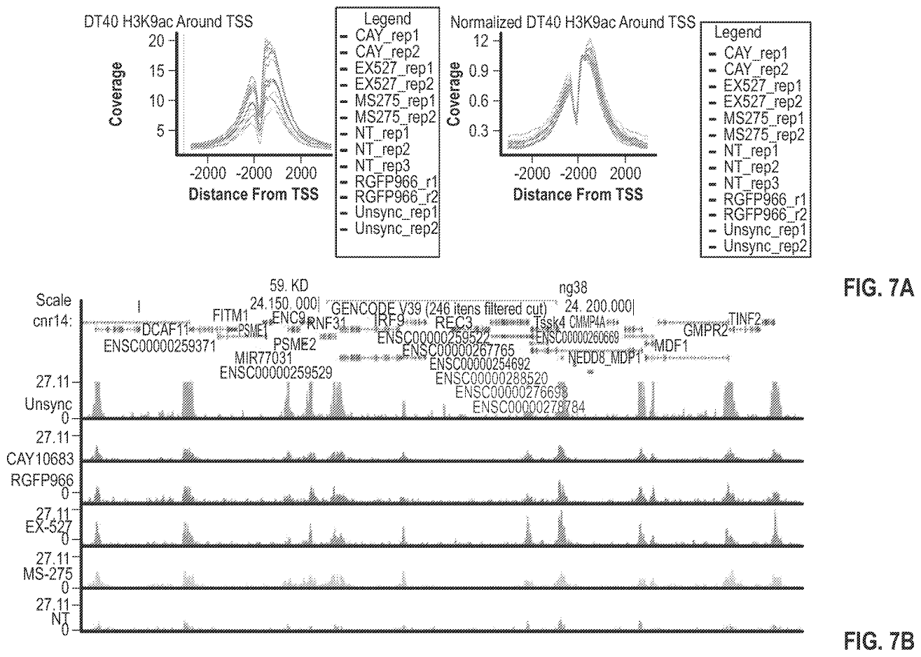

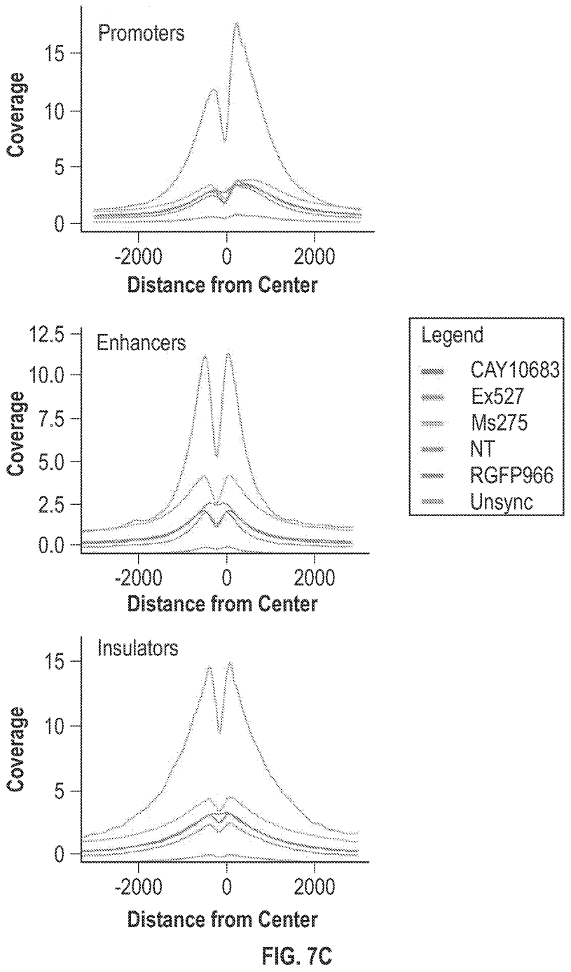

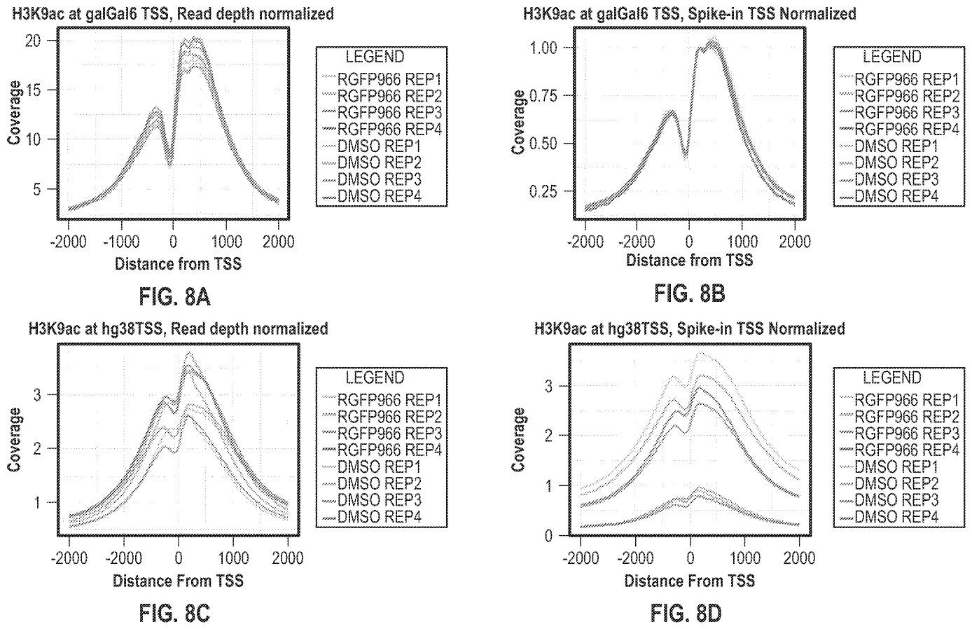

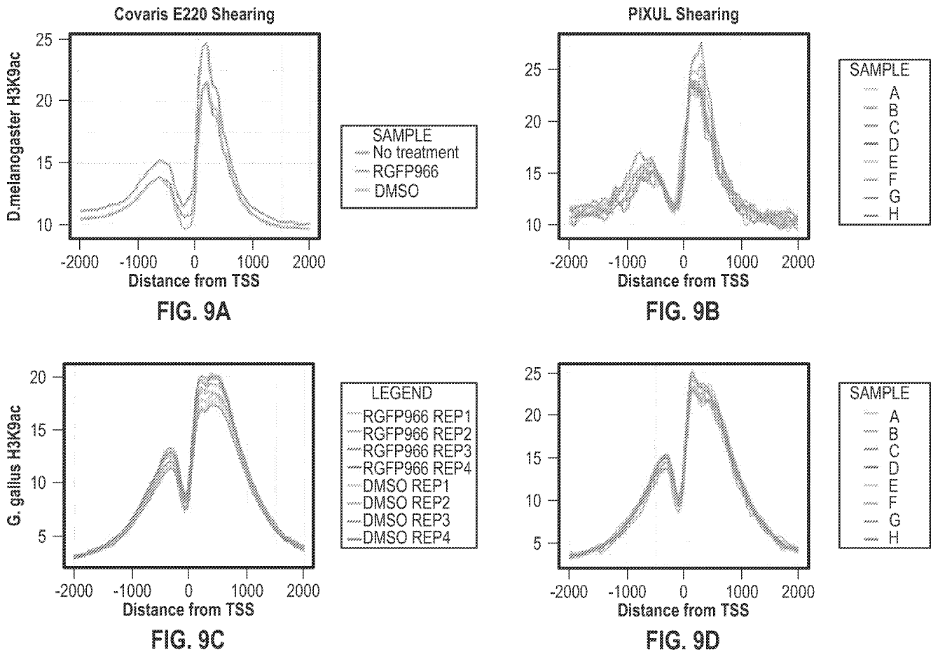

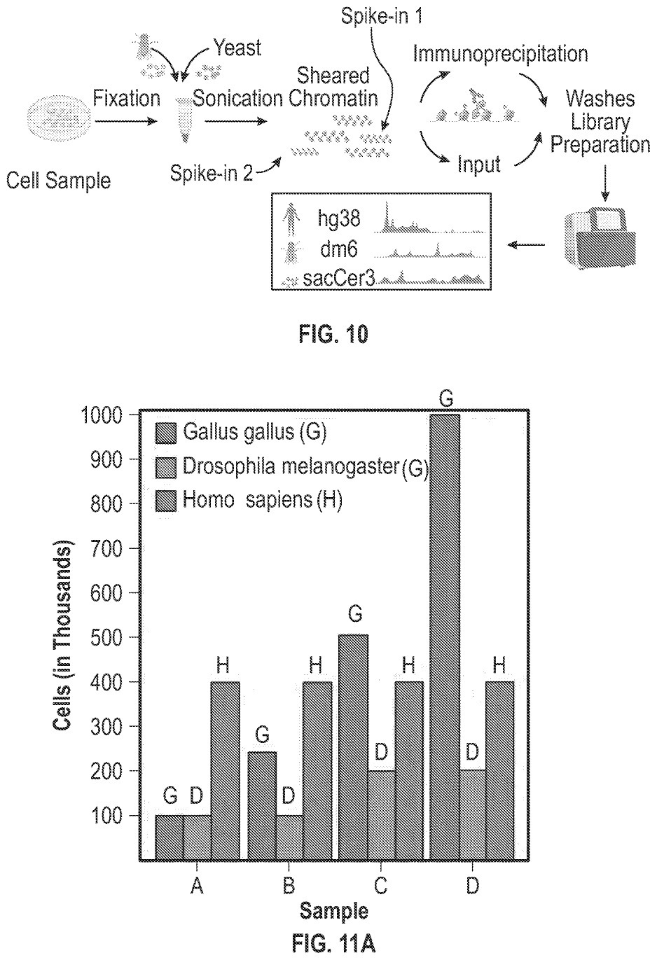

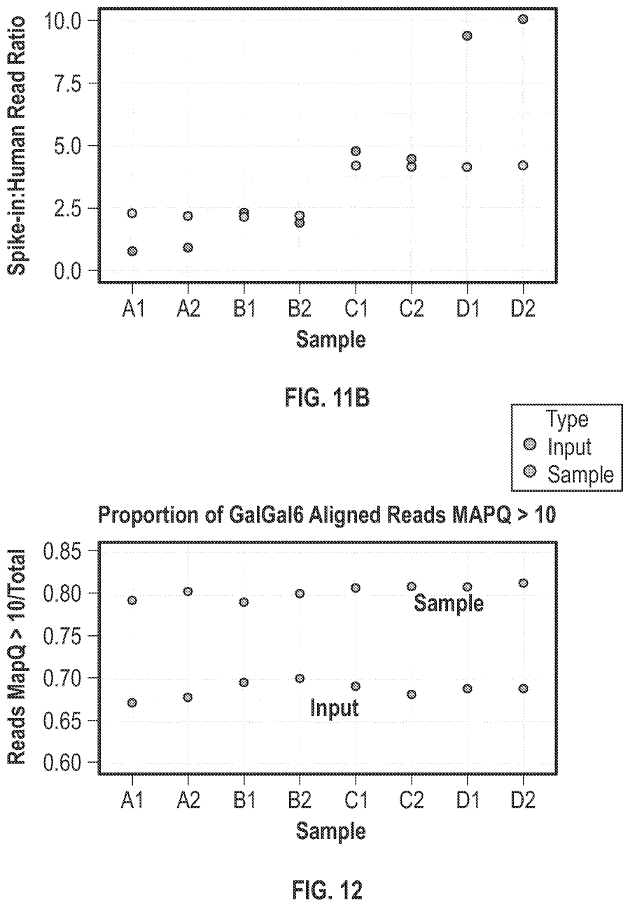

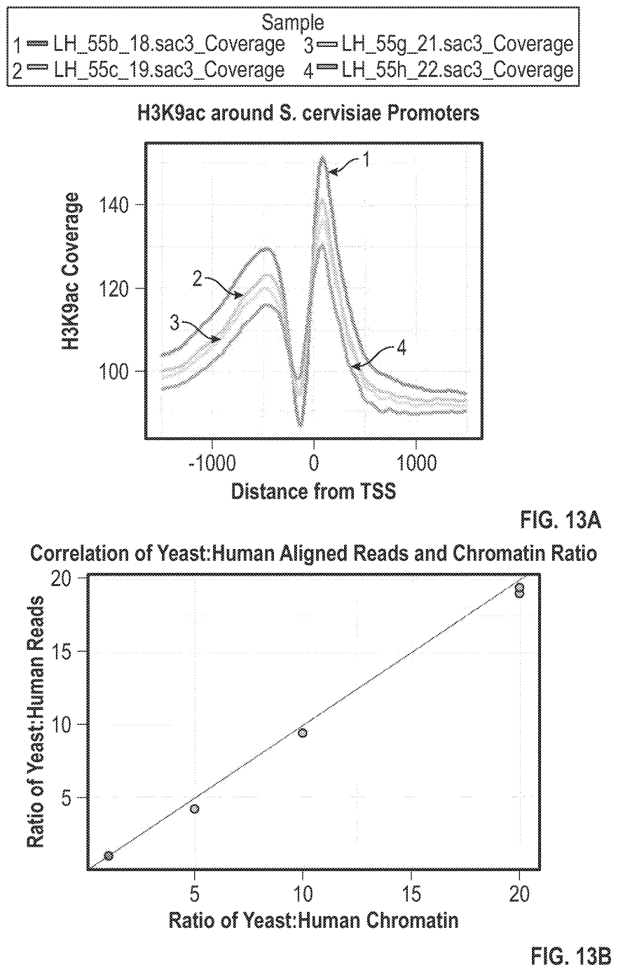

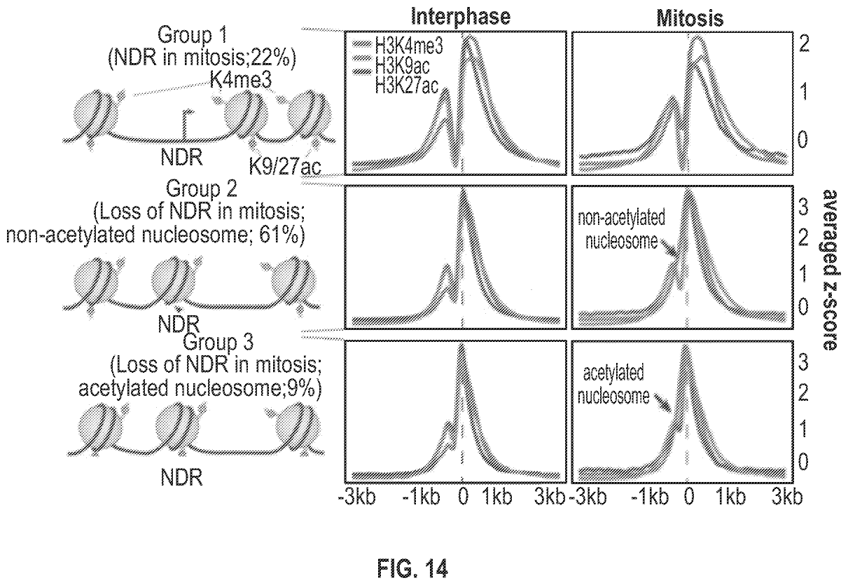

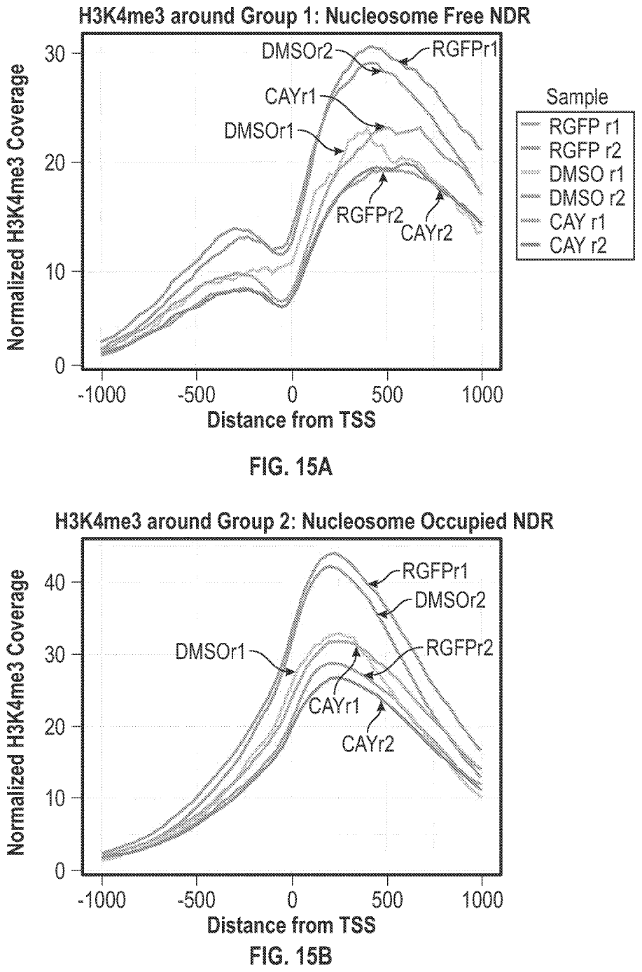

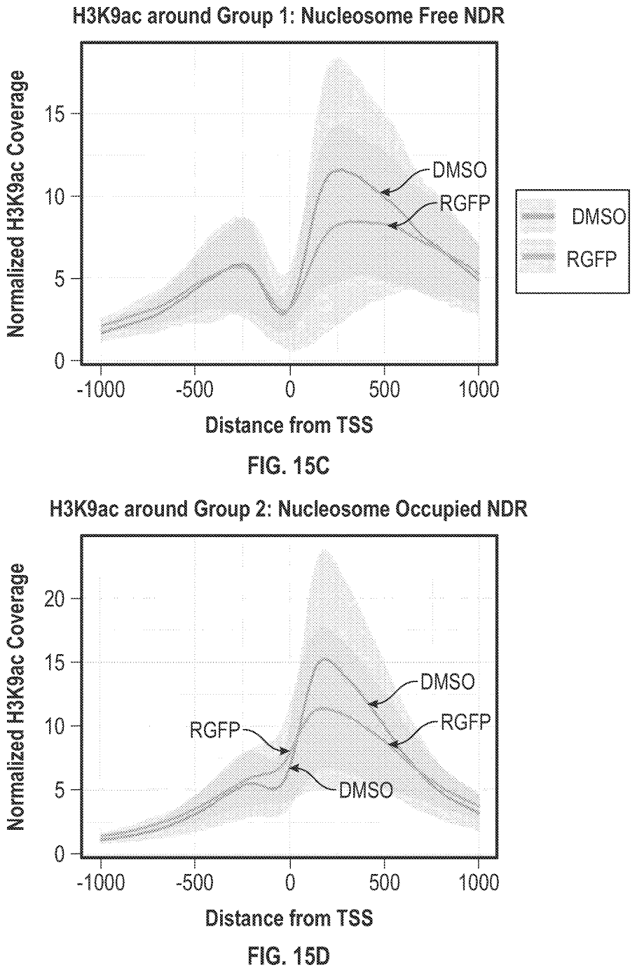

To benchmark our normalization method, we establish a ‘true positive’ by mixing mitotic-arrested (i.e., low acetylation) and interphase (i.e., high acetylation) cells in six concentrations—where our normalization is expected to accurately recover the relative acetylation levels in each mix. Without spike-in normalization global changes are often minimized; normalizing to the spike-in signal reveals the change in acetylation (FIG. 5). Similar to ChIP-Rx9, we see that prior to spike-in normalization only the condition with minimal H3K9ac (0% interphase) is different from the other conditions. The signal was normalized first by the ratio of spike-in input to account for variation in chromatin amounts, then normalized to the spike-in IP to account for antibody competition. After normalization, the replicates cluster more closely together and differences in acetylation between conditions are observable, and closely follow a linear pattern (FIG. 5, inserts).

### Automation of ChIP-Seq

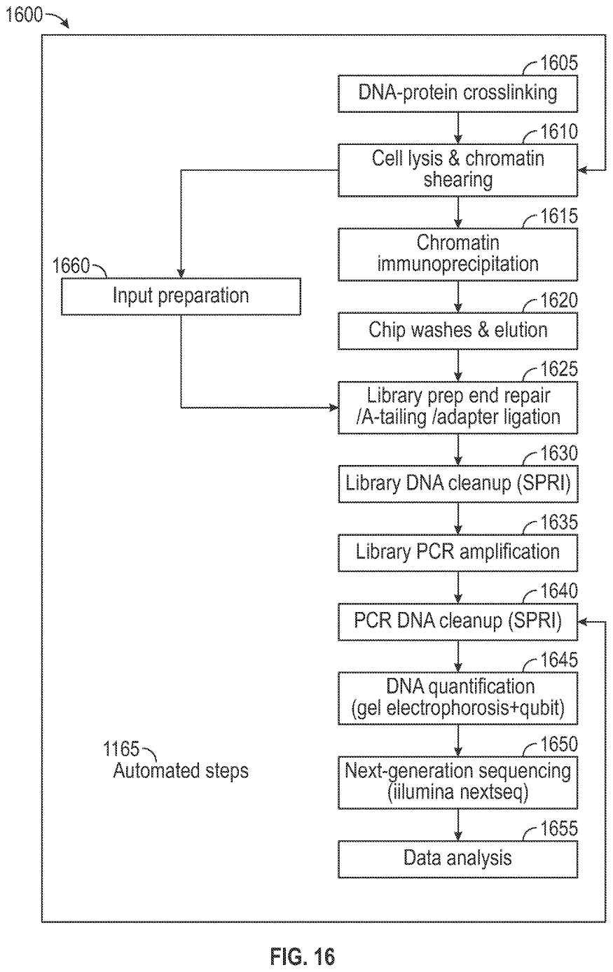

As described above, ChIP-seq is highly useful to elucidate where transcription regulators bind to regulate transcription in chromatin. However, the overall experimental ChIP-seq workflow involves multiple steps that increase the risk of introducing inconsistency within experiments and between groups (FIG. 16). These challenges can be partially addressed by the incorporation of automated liquid handling platforms to improve the robustness of the process (Blecher-Gonen et al., 2013; Busby et al., 2016). Yet, most of the previous efforts have automated only a subset of the ChIP-seq steps, such as only the bead cleanup steps or only the library preparation steps. Additionally, the majority of the ChIP-seq protocols—both automated and manual—are limited in their ability to efficiently map non-histone DNA-associated proteins, such as chromatin regulators that may indirectly interact with the genome (e.g., by being recruited to modified histones or histone variants).

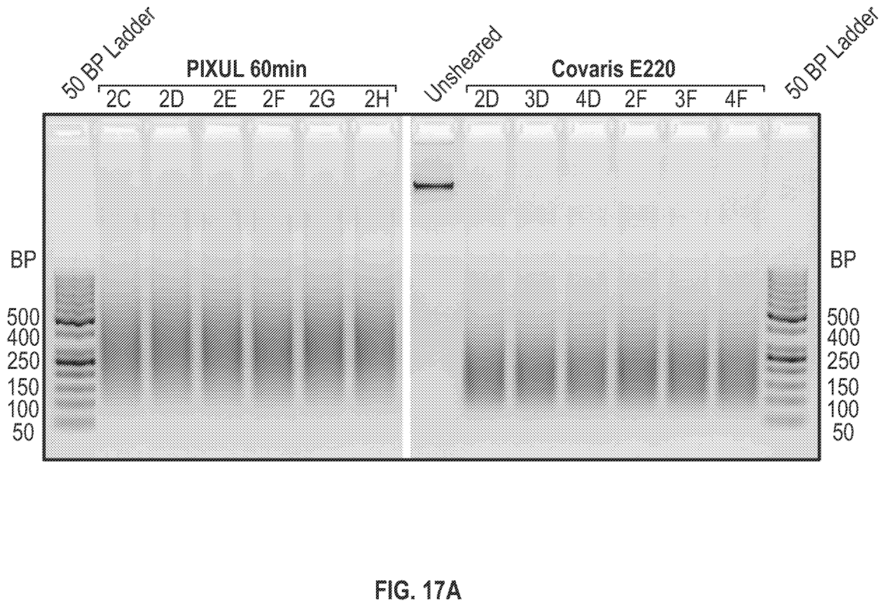

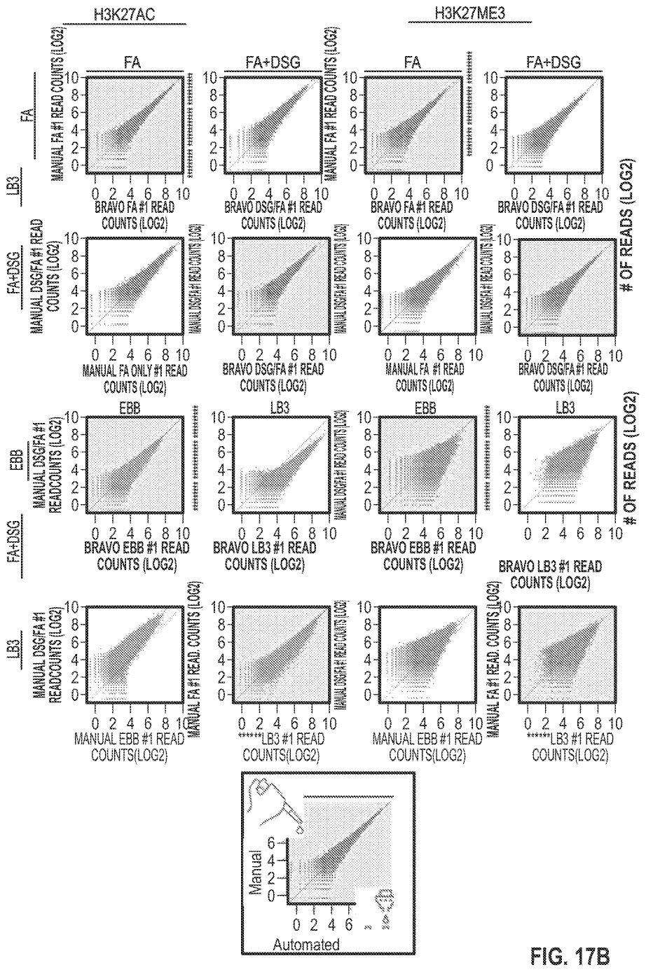

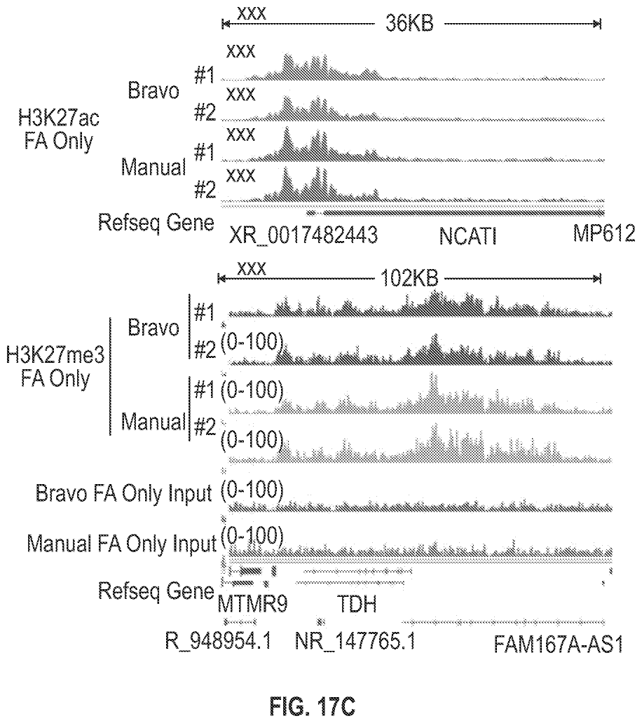

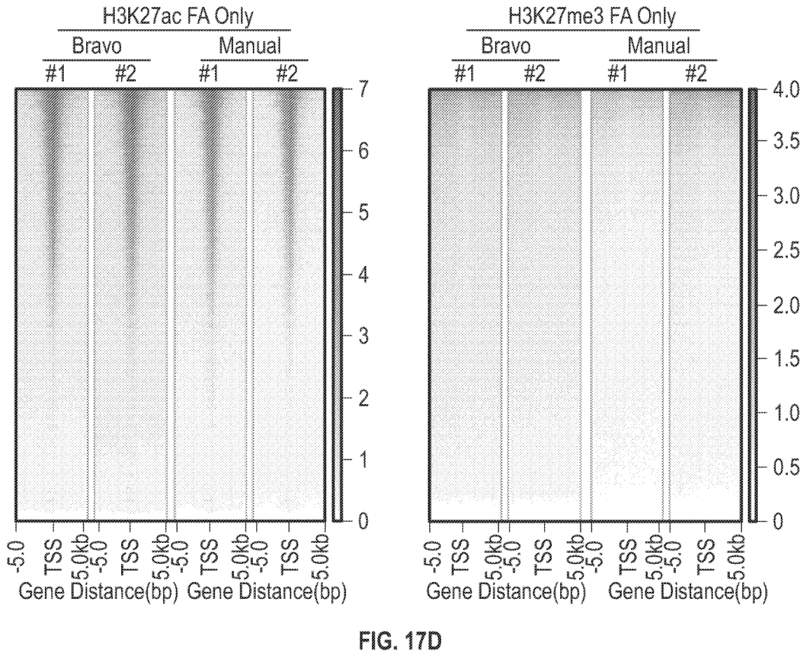

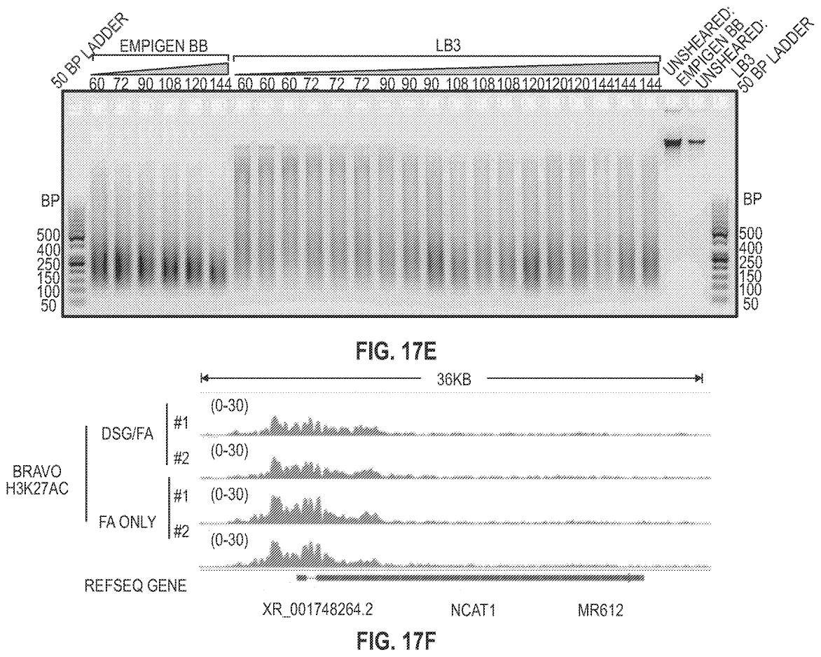

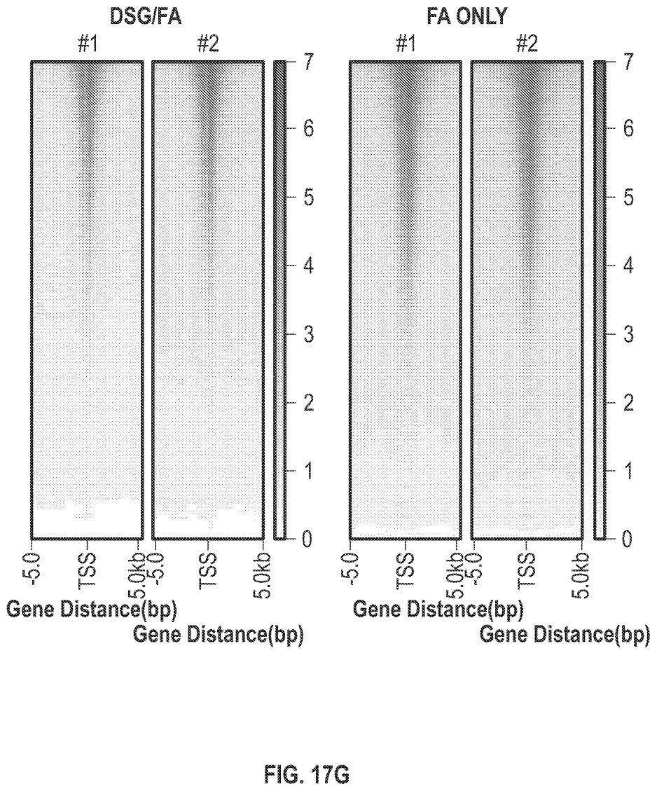

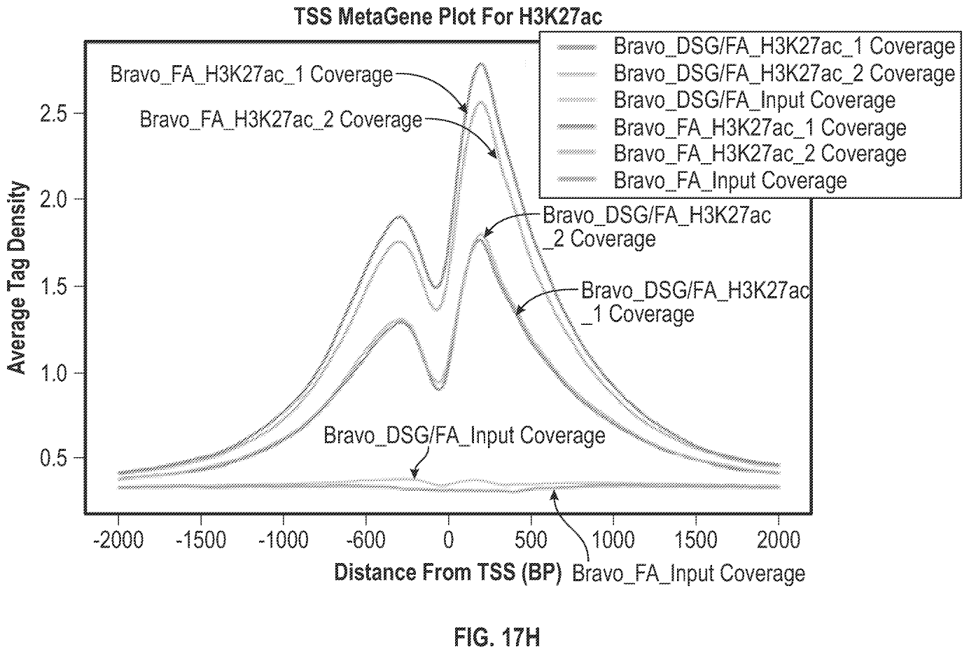

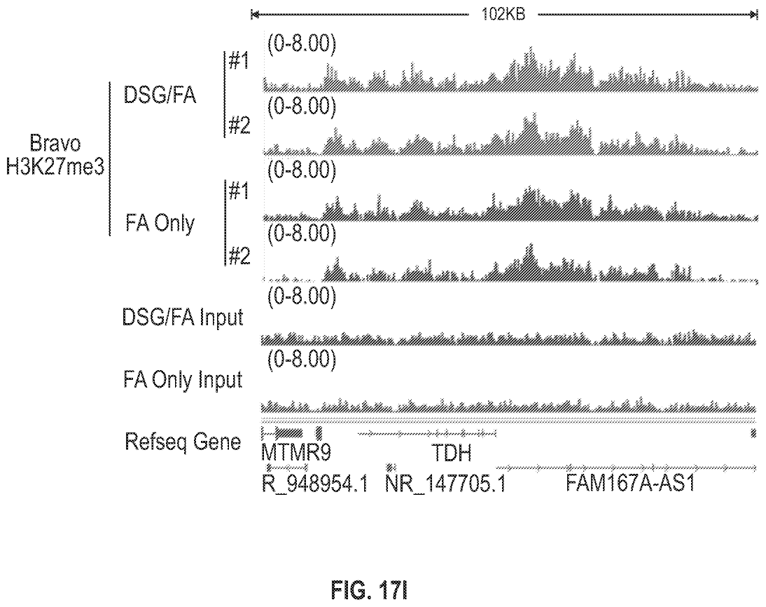

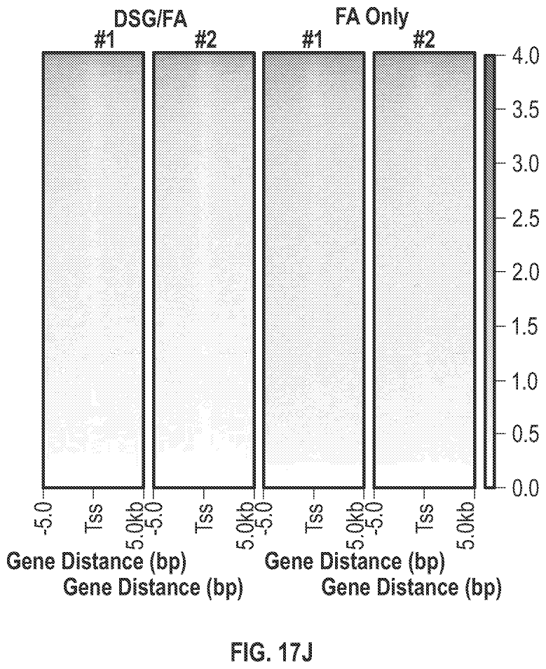

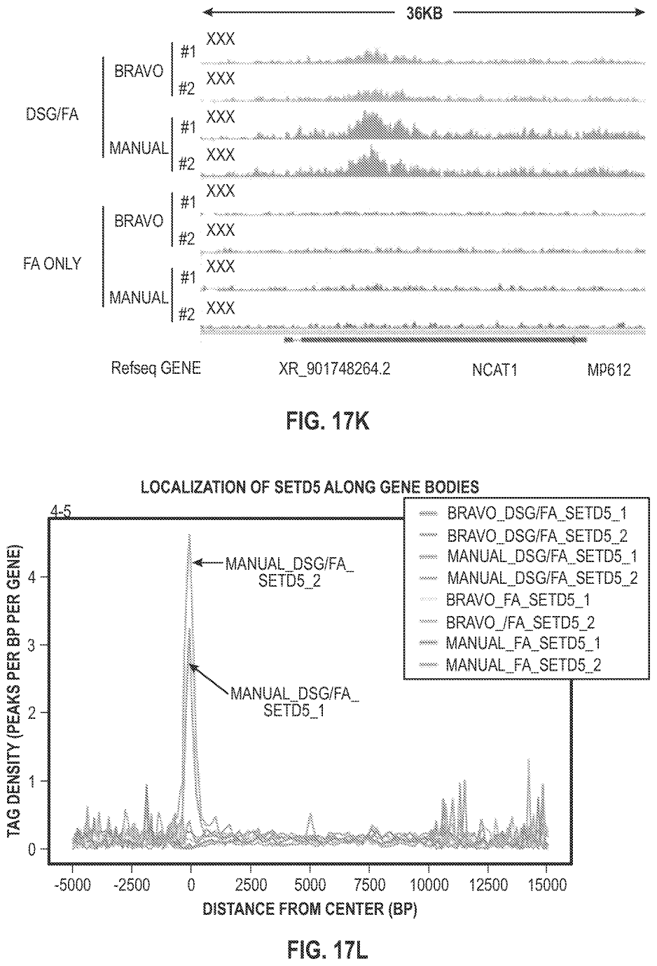

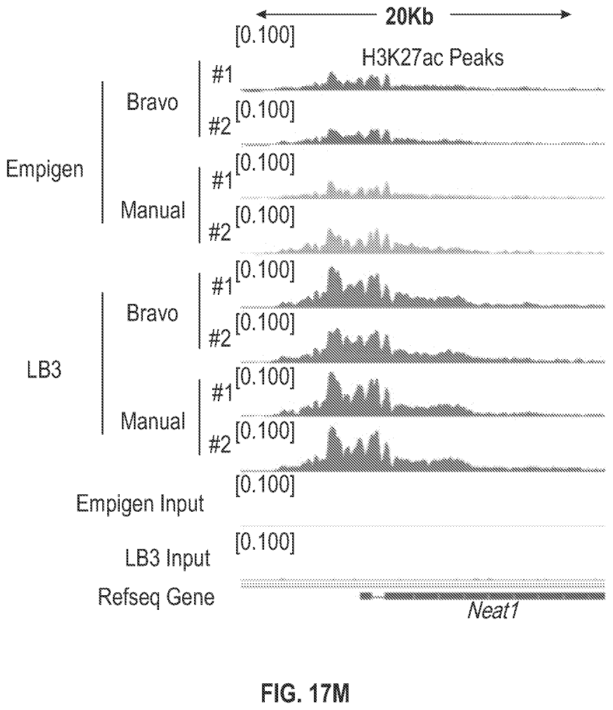

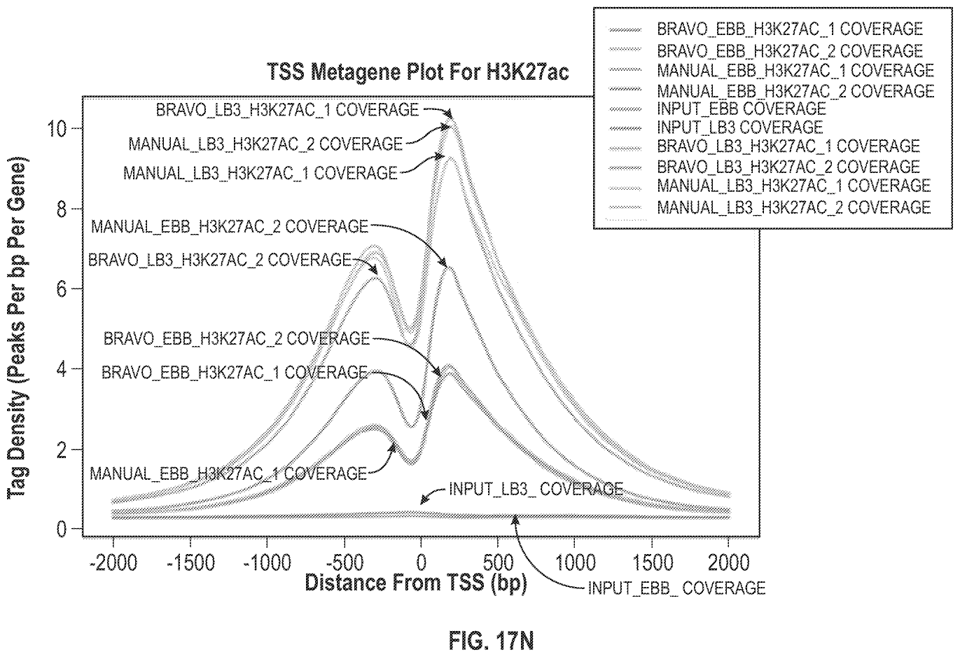

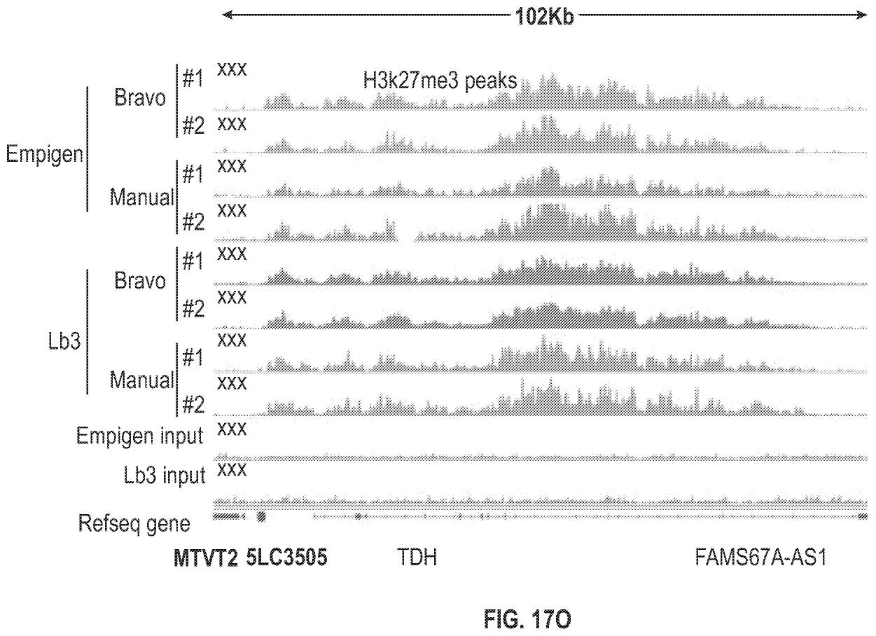

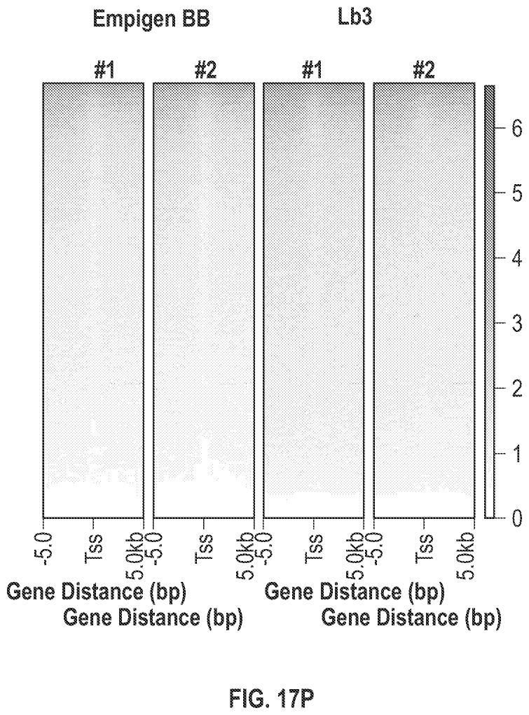

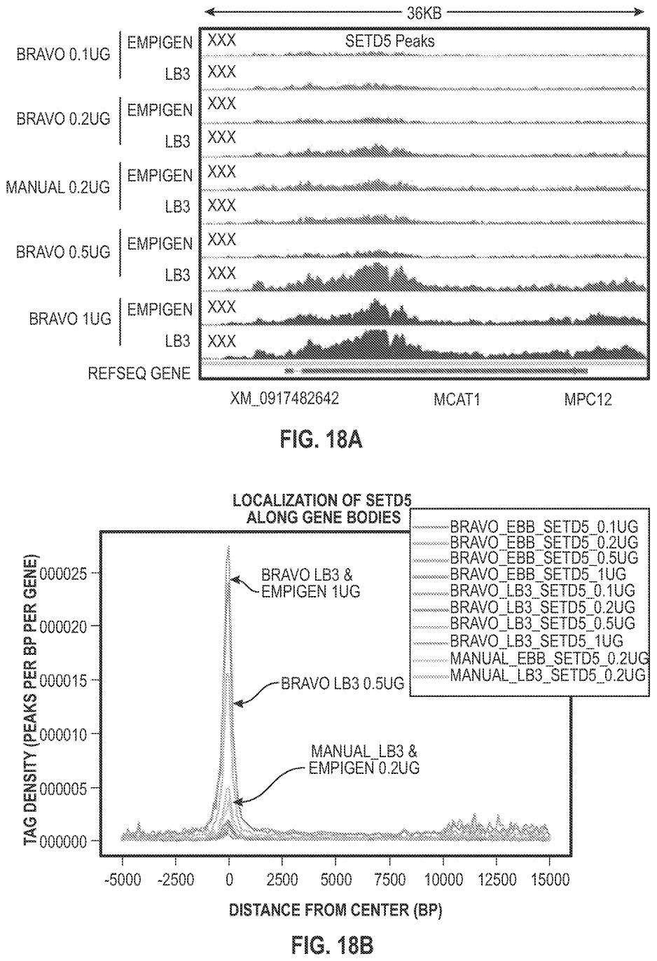

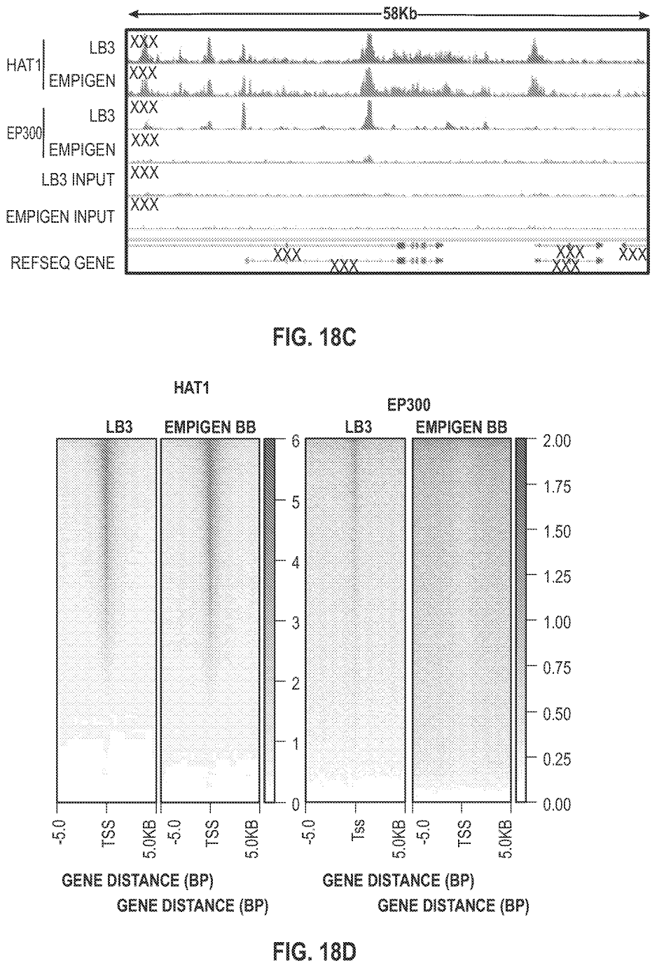

Methods for automation of ChIP-seq provided herein include a single-pot ChIP-seq protocol that incorporates an on-bead library preparation step adapted to be operated by a liquid handler to provide an end-to-end fully automated ChIP-seq method (FIG. 16). The automated ChIP-seq method includes an option for double crosslinking that increases the efficiency of detecting the genomic localizations of proteins that are indirectly associated with the DNA. In addition to reducing the workflow time, the automated ChIP-seq method also enables generation of sequencing libraries from low amounts of cell inputs. The automated ChIP-seq method is scalable and can conduct between 8-96 ChIP-seq reactions (e.g. in increments of 8). The ChIP-seq method is benchmarked by various reactions and demonstrated a high reproducibility and nearly indistinguishable signal-to-noise ratio between manual and automated workflows. (FIG. 17B-17D)

For example, the automated ChIP-seq method was used to evaluate multiple parameters, including shearing and crosslinking conditions, buffer compositions, and antibody ratios. (FIGS. 17A and 17B). Two different cell types were used: the aneuploid cervical cancer cell line HeLa-S3 and human induced pluripotent stem cells (hiPSCs) that has human induced pluripotent stem cells (hiPSCs) that has a normal karyotype. Optimal conditions were identified for more than a dozen DNA-associated proteins (including chromatin regulators and transcription factors). As a well validated reference, multiple key histone modifications were included that are associated with open and closed chromatin, and transcribed regions. (FIG. 17B).

An example of an automated ChIP-seq method 1100 is described in FIG. 16. The details protocols of each step in the automated ChIP-seq method 1600 are provided below in Example 1: Methods. The automated ChIP-seq method 1600 comprises automated steps 1665, which comprise steps 1610 through 1640. A sample comprising genomic DNA and proteins can be crosslinked 1605, such as with formaldehyde and/or disuccinimidyl glutarate (DSG). The crosslinked sample will contain crosslinked chromatin-protein complexes. The sample can be, for example, a cell culture. The crosslinked sample(s) can be loaded into any programmable liquid handling device. The liquid handler used for the experiments described herein is the Bravo liquid handler from Agilent Technologies. The cells can be lysed, and the chromatin sheared by any suitable method such as sonication 1610. The chromatin can be sheared to short fragments (e.g. 100-300 bp).

The sheared chromatin fragments can then be immunoprecipitated 1615 with an antibody that binds to chromatin or a protein known to bind chromatin. An input sample is obtained from a small subset of the chromatin lysate (~100 k cells) and serves as a non-IP control for background noise. The chromatin fragments of interest are then immunoprecipitated using antibodies specific to the DNA-binding protein under consideration or to a specific histone modification before the cross-links are reversed to obtain the DNA fragment population associated with either a given DNA-binding protein or a given modified histone, respectively. For example, the chromatin can be immunoprecipitated with an anti-SETD5 antibody for (Su(var) 3-9,E(z),Trithorax) “SET” domain containing proteins. The protein SET domain-containing 5 (Setd5) interacts with the polymerase-associated factor 1 complex (PAF1) co-transcriptional complex and has been shown to essential for regulating histone acetylation during gene transcription. Osipovich, A. B., et. al., Development. 2016 Dec. 15; 143(24):4595-4607. The ChIP is washed and eluted to produce the crosslinked chromatin-protein complexes 1620.

The crosslinked chromatin-protein complexes are used to generate ChIP-Seq libraries from ChIP-derived DNA 1625. Sequencing library is also made from the input sample. The sequencing library will determine the distribution and abundance of DNA-bound protein targets of interest across the genome. The sequencing library can be multiplexed DNA-barcode sequencing to allow pooling of multiple library preparations in a single flow cell lane for high throughput automation, reducing sequencing errors, and differentiating between samples.

The samples are reversed crosslinked with Proteinase K to digest proteins, leaving the nucleic acids. The library is then cleaned, such as with a Solid Phase Reversible Immobilization (SPRI) method of purifying nucleic acid from a solution using paramagnetic beads 1630. The library is amplified using polymerase chain reaction (PCR) methods 1635. The nucleic acid products of the PCR are then cleaned, such as by SPRI methods 1640.

The resulting amplified ChIP-seq libraries can be quantified, such as by gel electrophoresis or fluorometry 1645. The quantified DNA libraries can then be sequenced 1650. Data analysis of ChIP-seq sequencing reads includes mapping the reads to the reference genome and subsequent identification of signals associated with protein-binding sites or attachments of modified histones 1655. The aligned reads can be counted over a number of bins covering the entire genome, and it is determined whether a given bin count is different from the background, where the latter is modeled using Poisson distribution or estimated from the control sample. Different, probabilistic, peak-finding algorithms have been implemented in many available and popular programs, such as but not limited to Erange, FindPeaks, MACS, QuEST, Useq (http://sourceforge.net/projects/useq/), Homer (http://biowhat.ucsd.edu/homer/chipseq/index.html), CisGenome, Fseq, SISSRs, Swembl, PeakSeq, Qeseq, CCAT, ChipDiff, SICER, RSEG, TPIC, and W-ChlPeaks.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to formula (I) are equally envisaged as being applicable to formula (1b).

The invention will be further described by the following non-limiting examples.

### EXAMPLES

### Example 1: Methods

### 1. Cell Culture Preparation

HeLa-S3 (ATCC CCL-2.2™) adherent cells were cultured in supplemented DMEM with GlutaMAX™ (DMEM, 4.5 g/L D-Glucose, 110 mg/L sodium pyruvate, 2 mM L-glutamine, 10% FBS, 1% Antibiotic-Antimycotic) in an incubator set to 37 °C with 95% Air, 5% C02. When the cells were grown to confluency, they were harvested by adding 0.25% trypsin.

### 2. Crosslink Cells

Double crosslinking: The harvested cells were first crosslinked with at least 1 mL of 2 mM DSG/PBS solution per 1 million cells at room temperature for 30 min while rotating the tube overhead at 8 rpm. Then the cell pellet was resuspended in 1% formaldehyde/PBS solution per 1 million cells and incubate at room temperature for 10 min while rotating the tube overhead at 8 rpm. The crosslinking reaction was quenched by adding 1/20th volume of 2.625 M glycine and 1/20th volume 10% BSA. The cell pellet was resuspended and stored in 0.5% BSA/PBS.

Single crosslinking: The harvested cells were crosslinked with 1% formaldehyde/PBS solution per 1 million cells and incubated at room temperature for 10 min while rotating the tube overhead at 8 rpm. The crosslinking reaction was quenched by adding 1/20th volume of 2.625 M glycine and 1/20th volume 10% BSA. The cell pellet was resuspended and stored in 0.5% BSA/PBS.

### 3. Chromatin Sonication

Per 1 million crosslinked cell pellets, resuspend in 100 μL lysis buffers: SDS lysis buffer [1% SDS, 50 mM Tris-HCl (pH 8.1), 10 mM EDTA, 1×protease inhibitor cocktail], LB3 [10 mM Tris-HCl (pH 7.5), 100 mM NaCl, 1 mM EDTA, 0.1% Deoxycholate, 0.5% Sarkosyl, 1×protease inhibitor cocktail], Empigen BB lysis [0.5% Empigen BB (Sigma), 1% SDS, 50 mM Tris-HCl (pH 7.5), 10 mM EDTA, 1×protease inhibitor cocktail](Métivier et al., 2003).

For PIXUL, 100 μL of lysate was loaded into each well (~10,000-5,000,000 cells per well) and the plate was sealed with a PCR plate pressure seal.

For Covaris LE220-plus, 20 μL of lysate was loaded into each well and the plate was sealed with an aluminum plate seal.

After the chromatin was sonicated, lysate sheared with SDS was diluted 10-fold with CDB [0.01% SDS, 1.1% Triton X-100, 1.2 mM EDTA, 16.mM Tris-HCl, pH 8.1, 167 mM NaCl, 1×protease inhibitor cocktail] and lysate sheared with Empigen BB was diluted 2.5-fold in EBB dilution buffer [20 mM Tris/HCl pH 7.4@20 °C, 100 mM NaCl, 0.5% Triton X-100, 2 mM EDTA, 1×protease inhibitor cocktail). EMPIGEN® BB (EBB) is a 30% aqueous solution of the detergent lauryl betaine.

### 4. Input Preparation

Manual workflow: 1% or 10 μL of each sonicated lysate was saved as input sample. Per input sample, 55 μL 10% Triton X-100, 68 μL of Elution Buffer(10 mM Tris pH8, 0.5% SDS, 5 mM EDTA, 280 mM NaCl), and 1 μL RNase A were added and incubated in a thermocycler for 15 minutes at 37 °C. 1 μL of Proteinase K was added to each input sample and incubated at 55 °C for 1 hour and then 65 °C for 30 min. To clean up the input sample, 122 μL of Speedbead mastermix [2 μL SpeedBeads 2 μL SpeedBeads+120 μL 20% PEG8000/1.5M NaCl (final concentration=8.5% PEG, 1M NaCl)] was added to each input and incubated at room temperature for 10 minutes. After each sample was washed twice with 80% EtOH and Speedbeads pellet was air-dried, the input sample was eluted with 20 μL of TT (0.05% Tween 20, 10 mM Tris pH 8.0, warmed to 55 °C).

BRAVO robotic liquid handling system (hereinafter “Bravo”): Input plate was prepared with 1% or 1 OL of each lysate sample, and per lysate sample, the buffer plate was prepared with 55 μL 10% Triton X-100, 68 μL of Elution Buffer (10 mM Tris pH8, 0.5% SDS, 5 mM EDTA, 280 mM NaCl), and 1 μL RNase A. The Buffer Plate was placed on Bravo deck 5 and the Sample Plate was placed on Bravo deck 8. Bravo protocol 1a. Input Preparation was run. Sample plate was incubated in a thermocycler for 15 minutes at 37 °C. 1 μL of Proteinase K was added to each input sample and incubated at 55 °C for 1 hour and then 65 °C for 30 min. While the sample was incubating, a Speedbeads mastermix plate was prepared with 122 μL of speedbead mastermix [2 μL SpeedBeads 2 μL SpeedBeads+120 μL 20% PEG8000/1.5M NaCl (final concentration=8.5% PEG, 1M NaCl)] and 20 μL of TT (0.05% Tween 20, 10 mM Tris pH 8.0, warmed to 55 °C) per sample added to each well in the mastermix plate. Ethanol Plate was prepared with freshly made 80% EtOH. After incubation, the sample plate was back on Bravo deck 8, Mastermix Plate on deck 5 and Ethanol Plate on deck 2. Bravo protocol 1b. Input Speedbead Cleanup was run to clean up the input samples. At the end of the protocol, each well should contain 20 μL of eluted input in TT buffer.

### 5. Chromatin Immunoprecipitation

Per each ChIP sample, 10 μL of Dynabeads™ Protein A was washed twice with equal volume of LB3 lysis buffer+ 1/9th volume of 10% Triton X-100 for LB3 samples, or EBB dilution buffer for Empigen BB samples. Antibodies used in these experiments are H3K27ac (Acetyl-Histone H3 (Lys27) (D5E4) XP® Rabbit mAb from Cell Signaling Technology), H3K27me3 (Tri-Methyl-Histone H3 (Lys27) (C36B11) Rabbit mAb from Cell Signaling Technology), and SETD5 (0.1 mg/mL SETD5 Polyclonal Antibody Catalog #PA5-57318 from Invitrogen).

Manual workflow: 1 μL of H3K27ac, 1 μL of H3K27me3, 1, 2, 5, or 10 μL of SETD5 antibody was added to each tube containing washed Dynabeads Protein A accordingly. 50-100 μL of sheared chromatin (~500 k cells) was aliquoted into each sample tube.

Bravo workflow: Dynabeads Protein A resuspended in lysis buffer and appropriate volume of μL of antibody (same amount as manual workflow) was transferred to Bead/Antibody Plate. PIXUL lysate plate was placed on deck 2 and Dynabead/Antibody Plate on deck 5. Bravo protocol 2. Chromatin+Beads+Antibody Bind was run to aliquot 50-100 μL of sheared chromatin into Beads/Antibody Plate.

The ChIP samples were incubated overnight on HulaMixer™ Sample Mixer at 4 °C in the cold room (rotating at 8 rpm).

### 6. ChIP Washes

Manual workflow: After overnight incubation, the sample was washed three times with WBI [10 mM Tris pH7.5, 0.7% DOC, 1 mM EDTA, 250 mM LiCl, 1×protease inhibitor cocktail], three times with WBIII [20 mM Tris pH7.5, 1% Triton X-100, 2 mM EDTA, 150 mM NaCl, 0.1% SDS, 1×protease inhibitor cocktail], and twice with TET [10 mM Tris pH7.5, 0.2% Tween-20, 1 mM EDTA, 1×protease inhibitor cocktail]. Each ChIP sample was eluted in 25 μL of TT

Bravo workflow: Wash+Elution Buffer plate was prepared by aliquoting 600 μL of WBI/WBIII buffer, 400 μL of TET, and 30 μL of TT per well. Wash+Elution Buffer Plate was placed on deck 5, ChIP Plate on deck 7 (on ring magnet). Bravo protocol 3. ChIP Wash+Elution Off Mag was run. At the end of the protocol, each well should contain 25 μL of eluted ChIP sample in TT buffer.

### 7. Library Construction

We used the NEBNext® Ultra™ II DNA Library Prep Kit for Illumina (Catalog #E7645L) and NEXTflex® DNA Barcodes—48 (NOVA-514104) for library construction.

Manual workflow. 2 μL of each input supernatant was transferred to a new tube and 23 μL of TT was added to make a total volume of 25 μL. (1) End Prep: Per each ChIP and input sample, 5 μL of End Prep Mastermix (1.5 μL of NEBNext Ultra II End Prep Enzyme Mix and 3.5 μL of NEBNext Ultra II End Prep Reaction Buffer) was added and incubated for 30 min at 20 °C, and then 30 min at 65 °C in a thermal cycler with the lid set to 75 °C. (2) Adapter ligation: The ChIP and input samples were recovered from the thermal cycler and 1 μL of NEXTflex-DNA-Barcodes-48 Bioo ChIP Adaptor (1:40 diluted 0.625 uM) was added to each sample. Per each ChIP and input sample, 15.5 μL of Ligation Mastermix [15 μL NEBNext Ultra II Ligation Master Mix and 0.5 μL of NEBNext Ligation Enhancer] was added and incubated for 15 mins at 20 °C with the heated lid set to “off.” After incubation, ligation reaction was stopped by adding 27 μL of ligation stop solution mastermix [4 μL 10% SDS+3 μL 0.5M EDTA+20 μL water] and 4.5 μL of 5M NaCl per sample.

Bravo workflow: 2 μL of each input supernatant was transferred to the ChIP plate and 23 μL of TT was added to make a total volume of 25 μL. (1) End Prep: Per sample, 5 μL of End Prep Mastermix was prepared with 1.5 μL of NEBNext Ultra II End Prep Enzyme Mix and 3.5 μL of NEBNext Ultra II End Prep Reaction Buffer. Mastermix was added to the Mastermix Plate and the plate was placed on deck 5. The ChIP Plate was placed on deck 8. Bravo protocol 4. End Repair MM Dispense was run to add 5 μL of End Prep Mix to each ChIP and input sample. The ChIP plate was incubated for 30 min at 20 °C, and then 30 min at 65 °C in a thermal cycler with the lid set to 75 °C. (2) Adapter ligation: Per sample, 15.5 μL of Ligation Mastermix was prepared with 15 μL NEBNext Ultra II Ligation Master Mix and 0.5 μL of NEBNext Ligation Enhancer. Mastermix was added to the Mastermix Plate and the plate was placed on deck 5. The ChIP plate was recovered from the thermal cycler and 1 μL of NEXTflex-DNA-Barcodes-48 Bioo ChIP Adaptor (1:40 diluted 0.625 uM) was added to each sample. The ChIP plate was placed back to deck 8. Bravo protocol 5. Adapter Ligation MM Dispense was run to add 15.5 μL of Ligation Mastermix to each sample. The ChIP plate was incubated for 15 mins at 20 °C with the heated lid set to “off.” Per sample, 27 μL of ligation stop solution mastermix [4 μL 10% SDS+3 μL 0.5M EDTA+20 μL water] and 4.5 μL of 5M NaCl was prepared as a Buffer Plate and the plate was placed on deck 5. After incubation, the ChIP Plate was placed back to deck 8 and Bravo protocol 6. Ligation Stop Solution Dispense was run to dispense the stop solution.

### 8. Reverse Crosslinking and Library Cleanup

The samples were reverse crosslinked by adding 1 μL of Proteinase K to each sample and incubating for 1 hour (or 30 mins for cells crosslinked with FA only) at 55 °C and then 30 mins at 65 °C.

Manual workflow: The supernatant of each sample was separated from Dynabeads Protein A and added to tubes containing 63 μL of Speedbeads mastermix [2 μL SpeedBeads+61 μL 20% PEG8000/1.5M NaCL (8.6% PEG, 0.8M NaCl)]. After 10 min of incubation at room temperature, each sample was washed twice with 200 μL of 80% EtOH. The Speedbeads pellet was air-dried and the sample was eluted with 25 μL of TT (0.05% Tween 20, 10 mM Tris pH 8.0, warmed to 55 °C).

Bravo workflow: Per sample, 63 μL of Speedbeads mastermix [2 μL SpeedBeads+61 μL 20% PEG8000/1.5M NaCL (8.6% PEG, 0.8M NaCl)] and 30 μL of TT (0.05% Tween 20, 10 mM Tris pH 8.0, warmed to 55 °C) was added to each well in the Buffer Plate. The Buffer Plate was placed on Bravo deck 5. The Ethanol Plate was prepared with 400 μL of 80% EtOH per sample and placed on deck 2. The ChIP Plate on Bravo deck 7 (on ring magnet). Bravo protocol 7. Adapter Ligation Cleanup was run. At the end of the protocol, each well should contain 25 μL of eluted sample in TT buffer.

### 9. PCR Amplification and Final Cleanup of ChIP and Input Libraries

The PCR mastermix was created by combining 25 μL NEBNext Ultra II Q5 2× Mastermix with 0.25 μL each of 100 μM sequencing platform-compatible forward and reverse primers (Solexa 1GA& Solexa 1 GB). The 25.5 μL of PCR mastermix was added to each sample either manually or by running Bravo protocol 8. PCR Enrichment MM Dispense with the Mastermix Plate on deck 5 and ChIP Plate on deck 7.

A PCR program was run to amplify the libraries using a thermal cycler with a heated lid set to 105 °C: Initial denaturation at 98 °C for 30 seconds; 12 cycles of denaturation at 98 °C for 10 seconds, annealing at 60 °C for 15 seconds, extension at 72 °C for 30 seconds; and final extension at 72 °C for 2 minutes.

Manual workflow: 40.5 μL of Speedbead mastermix [2 μL SpeedBeads+38.5 μL of 20% PEG/2.5M NaCl (final concentration=8.5% PEG, 1M NaCl)] was added to each sample and incubated at room temperature for 10 minutes. After each sample was washed twice with 80% EtOH and Speedbeads pellet was air-dried, the input sample was eluted with 15 μL of TT (0.05% Tween 20, 10 mM Tris pH 8.0, warmed to 55 °C).

Bravo workflow: Speedbeads mastermix plate was prepared with 40.5 μL of Speedbead mastermix [2 μL SpeedBeads+38.5 μL of 20% PEG/2.5M NaCl (final concentration=8.5% PEG, 1M NaCl)] and 30 μL of TT (0.05% Tween 20, 10 mM Tris pH 8.0, warmed to 55 °C) per sample added to each well in the mastermix plate. Ethanol Plate was prepared with freshly made 200 μL of 80% EtOH. The sample plate was placed on Bravo deck 7 (on ring magnet), Buffer Plate on deck and Ethanol Plate on deck 2. Bravo protocol 9. PCR Enrichment Cleanup was run to clean up the input samples. At the end of the protocol, each well should contain 15 μL of eluted sample in TT buffer.

### 10. DNA Quantification and Sequencing

DNA size quantification: 5 μL of each library was taken out and mixed with 1 μL of 6× TriTrack DNA Loading Dye to run on a 2% agarose gel pre-stained with GelGreen.

DNA concentration quantification: Qubit HS DNA assay buffer and standards were used to estimate the concentration of the libraries. The libraries were pooled according to the concentration calculations. The pooled libraries were prepared and sequenced with Illumina NextSeq as single reads (64 or 84 bp).

### 11. Sequencing Data Analysis

Sequencing reads were mapped to human reference genome hg38 using Bowtie2 (Langmead & Salzberg, 2012) with default parameters and duplicate reads were removed with SAMtools (Li, et al., 2009). HOMER (Heinz, et al., 2010) was used to make tag directories and bigwig files for visualization on IGV genome browser (Robinson et al., 2011). Peaks were called with HOMER findPeaks program with parameter “-style histone.” Metagene plots centered on TSS was made with HOMER annotatePeaks.pl program set in “TSS” mode. Metagene plots along the gene bodies were generated with HOMER makeMetaGeneProfile.pl program set in “ma” mode. Correlation scatterplots were generated using HOMER annotatePeaks.pl program centered on merged peak file from all samples for each antibody (made with mergePeaks program) to quantify the read counts from each experiment at each of the peaks. Both HOMER programs automatically normalize each tag directory by the total number of mapped reads.

### Example 2: Histone Deacetylation During Mitosis

Mitosis is a well-studied phenomena where cells temporarily reorganize their epigenome, while allowing for identity and function to be properly relayed to daughter cells. While the levels of histone methylation remain relatively stable during mitosis, histone acetylation is globally reduced. Further, we observed that a nucleosome that is specifically deacetylated relative to neighboring nucleosomes enters the nucleosome depleted region (NDR) of many promoters during mitosis. Treatment of mitotic cells by an HDAC inhibitor abrogates this pattern, and this nucleosome becomes acetylated.

Here, we study the mechanisms regulating mitotic deacetylation of the nucleosomes entering the NDRs, and the general contributions of HDACs to histone deacetylation during mitosis. We focus on H3K9ac, a histone modification that is highly depleted in mitosis, both globally and on the NDR localized nucleosome. We observe that HDAC2 and HDAC3 are playing a key role in modulating mitotic H3K9ac. Further, we and others have shown that key HATs and HDACs localize to NDRs, in line with the reversible deacetylation of nucleosomes located in the NDR during mitosis.

We follow these results and use automated ChIP-seq to map the genomic localization of HAT1, EP300 and H3K9ac following specific inhibition of the HDACs. To accurately quantify the ChIP-seq signal following the perturbations, we have developed a novel normalization strategy that improves on existing spike-in methods. We include different ratios of exogenous chromatin samples from two distinct species. The two spike-ins allow us to achieve a robust normalization that is based on the known linear relationship between the two exogenous chromatin signals. We demonstrate that specific inhibition of HDAC2 and/or HDAC3 increases H3K9ac in mitotic cells. Our preliminary results show that HDAC3 is associated with the mitotic deacetylation of the nucleosomes entering the NDR. Together, we show that HDAC2 and HDAC3 are associated with the reduced levels of H3K9ac during mitosis, we identify specific genomic loci that are bound by HDAC2, HDAC3, HAT1 and EP300 during mitosis and interphase and we present a novel, highly precise ChIP-seq normalization approach.

### Example 3: Automated ChIP-Seq Method

Evaluation of sonicators:

In previous work (Busby et al), we used Covaris E220 for shearing up to 96 samples. As the Covaris E220 device sonicates only one sample at a time, shearing 96 samples requires approximately 10 hours. Recently, a new sonication device, PIXUL (Bomsztyk et al., 2019) provided the ability to shear 96 samples in only 1-2 hours. We decided to evaluate the ability of PIXUL to reproducibly shear samples in a manner that is at least comparable to the Covaris E220. For both devices we additionally tested the impact of placing samples at different locations within the 96-well plate (FIG. 17A). In addition to the sonicators, we also evaluated two lysis buffers. For each lysis buffer we compared shearing pattern and signal-to-noise ratio in the obtained ChIP-seq results.

Establishment and evaluation of a single-pot automated ChIP-seq protocol:

We built on our previous automated ChIP-seq set up (Busby) and modified the parameters to accommodate our recent single-pot ChIP-seq protocol (Texari). The specific settings of the liquid handler require optimization, and for instance, aspirating and dispensing speeds should be adequate for the viscosity of each specific buffer. Thus, during the initial optimization steps, we assessed the performance of the automated ChIP-seq protocols by benchmarking it against the manual ChIP-seq using the same input material. To conduct this benchmarking, we focused on both H3K27ac, a histone modification that is associated with open chromatin, and H3K27me3, a histone mark that is linked to repression of broad genomic regions. At the same time, we also compared the impact of single and double crosslinking conditions as well as buffer compositions (detailed below). We used PIXUL to shear ~1M crosslinked HeLa-S3 cells/well; to reduce variation, following the shearing step we merged and then split the lysate from all wells between the two conditions (manual or automated ChIP-seq). Additionally, we used the same setup to compare the effectiveness of two different lysis buffer compositions, namely EBB and LB3. In total, for this initial evaluation, we performed 32 ChIP-seq experiments, evaluating 16 different conditions, each in duplicates. Together, the ChIP-seq results for the histone modifications H3K27ac and H3K27me3 were highly concordant between the manual and automated for variable conditions we tested, validating the liquid handler settings we optimized (FIG. 17B-17D).

Systematic evaluation of crosslinking conditions and buffer compositions:

The conventional way of crosslinking involves a single step of formaldehyde (FA) to create covalent bonds between protein and DNA. However, FA is a small molecule and only captures interactions in the range of 2.3-2.7 Å, which limits the capturing distance of protein interactions. Thus, to enable detection of protein interactions occurring between molecules that are further apart, larger FA-compatible crosslinkers, such as disuccinimidyl glutarate (DSG) that can form crosslinks of approximately 7.7 Å, are combined with FA. The double crosslinking process first uses DSG to stabilize multiple-protein complexes, followed by FA treatment for protein-DNA crosslinking reaction.

We observed that the samples treated with the additional crosslinker need a longer sonication/shearing time. (FIG. 17E). Thus, we attempted to use EMPIGEN® BB (EBB), which was previously shown to potently assist the shearing process and compare it with Lysis Buffer3 (LB3). Sonication of the double crosslinked samples in EBB for 60 minutes provided optimal fragmentation patterns. While initially the fragmentation of double crosslinked samples in LB3 buffer was inefficient and resulted in residual high molecular weight fragments, increasing the sonication time to ~90-108 minutes resulted in optimal shearing patterns (data not shown). Next, we used the double crosslinked samples and performed ChIP-seq of the histone modifications Histone H3 Lysine 27 acetylation (H3K27ac) and histone 3 lysine 27 trimethylation (H3K27me3), as well as the chromatin regulators histone acetyltransferase 1 (HAT1) and E1A binding protein p300 (EP300) and either lysed the cells with EBB or LB3. Overall, we observed that the samples sheared in EBB had lower signal-to-noise ratio (SNR) compared to the ones sonicated in LB3 potentially due to the high sodium dodecyl-sulfate (SDS) levels in the EBB buffer (1% SDS; FIGS. 17B-17D).

Given the low SNR we obtained when using EBB—both for mapping modified histones and non-histone DNA-associated proteins, we conducted the rest of the experiments only with the LB3 buffer and sonicated the double crosslinked samples for 108 minutes. Using these conditions, the H3K27me3 and H3K27ac ChIP-seq results from the double crosslinked samples were mostly indistinguishable from the single crosslinked ones, providing evidence that the additional crosslinker or longer shearing did not introduce bias in either active or repressed chromatin environments (FIGS. 17B-17D). These results support the use of the double crosslinking for mapping non-histone DNA-associated proteins. Optimization of conditions for genomic mapping of non-histone DNA-associated proteins:

Next, we used the ChIP-seq conditions identified above to map non-histone DNA-associated proteins in double crosslinked HeLa-S3 cells. As an example, we focused on 8 DNA-associated proteins (namely transcriptional repressor CTCF (Uniprot No. Ref. P49711), Histone acetyltransferase p300 (EP300) (Uniprot No. Ref Q09472), Histone acetyltransferase type B catalytic subunit (HAT1) (Uniprot No. Ref. 014929), Histone deacetylase 3 (HDAC3) (Uniprot No. Ref. 015379), Nuclear respiratory factor 1 (NRF1) (Uniprot No. Ref. Q16656), DNA-directed RNA polymerase II subunit RPB1 (POLR2A) (Uniprot No. Ref. P24928), SET domain-containing 5 (SETD5) (Uniprot No. Ref. Q9COA6), and Yes-associated protein (YAP1)(Uniprot No. Ref P46937) to perform ChIP-seq, each in duplicate. Following our previous efforts to increase ChIP-seq standardization, we aimed to mainly focus on monoclonal antibodies and used our automated approach to evaluate between 2-4 different antibodies. Together, we were able to map 6 of the 8 DNA-associated proteins we tested, although some of the antibodies were not able to generate a signal under the conditions we tested.

### Example 4: Spike-Ins from Same Cell Type as Cells Tested

In some embodiments, a controlled and calibrated experiment can be generated using the dual spike-in method wherein the dual spiked-in DNA is from the same cell type to the one that is being tested (eg treatment vs control of human induced pluripotent stem cells (hiPSCs)-derived neurons) (See description of FIG. 10). To do that, the DNA is barcoded while it is still organized as chromatin—this way the same cells population can be used with different barcodes and each barcoded chromatin at a different ratio. There are established methods for labeling the DNA while it is in a chromatin phase. In previous studies, these methods used the same chromatin for two consecutive immunoprecipitaton steps (e.g. co-ChIP https://www.nature.com/articles/nbt.3652). This had some additional advantages for ChIP-seq normalization over using dual species spike ins, such as (i) Avoiding the issue of missing epitopes in some of the species for some antibodies (i.e. when a target is not present in yeast); (ii) it may allow one to run treatment and control chromatin through the same IP reactions etc. and thus eliminate some of the technical variation in the assays, etc. Sort of allows you to do ‘single pot’ assays where all the samples are included together.

### Statements

The following statements are intended to describe and summarize various embodiments of the technology according to the foregoing description in the specification.

1. A method for normalizing chromatin immunoprecipitation (ChIP) sequence data comprising:
obtaining a culture of animal cells;
arresting a cell cycle of the animal cells;
crosslinking the animal cells;
harvesting the animal cells;
sonicating the animal cells to produced sheared chromatin;
   spiking the sheared chromatin with at least two exogenous chromatin samples from two species that are different from the species of the animal cells to produce a combined chromatin sample, wherein the ratio of the amount of the at least two exogenous chromatin samples is known;
immunoprecipitating the combined chromatin sample;
producing a ChIP-seq library;
amplifying the ChIP-seq library;
sequencing the immunoprecipitated combined chromatin sample to produce sequencing data; and
   normalizing the sequencing data based on the known linear relationship between the at least two exogenous chromatin samples.
   2. The method of statement 1, wherein the animal cells are arrested in a prometaphase-like state.
   3. The method of statement 1, wherein the animal cells are crosslinked in Dimethyl pimelimidate (DSG) with phosphate buffered saline (PBS), formaldehyde, or both.
   4. The method of statement 1, wherein the ratio of the amount of the at least two exogenous chromatin samples is different.
   5. The method of statement 1, wherein an antibody used in immunoprecipitating the chromatin recognizes an
epitope in the chromatin and in the at least two exogenous chromatin samples.
   6. The method of claim 1, wherein normalization is quantitative.
   7. The method of statement 1, wherein a liquid handler is used in at least one of the steps of sequencing, immunoprecipitating, producing the ChIP-seq library, amplifying, or normalizing.
   8. The method of statement 1, wherein the chromatin in the at least two exogenous chromatin samples are sheared.
   9. The method of statement 1, wherein DNA in the at least two exogenous chromatin samples from the same species as the animal cells are barcoded while the DNA is still organized as chromatin.
   10. A method of using an automated liquid handler to conduct ChIP-seq reactions comprising:
obtaining a culture of animal cells;
crosslinking the animal cells;
harvesting the animal cells;
sonicating the animal cells to produced sheared chromatin;
input the sheared chromatin into a sample plate in an automated liquid handler;
actuate the automated liquid handler to at least one of:
   immunoprecipitate the sheared chromatin;
   wash and elute the immunoprecipitated sheared chromatin;
   prepare a library;
   amplify the library;
   quantify DNA in the library;
   sequence the library.
      11. The method of statement 10, wherein a cell cycle of the animal cells is arrested before crosslinking the animal cells.

The specific methods, devices and compositions described herein are representative of various embodiments and are exemplary and not intended as limitations on the scope of the technology. Other objects, aspects, and embodiments will occur to those skilled in the art upon consideration of this specification and are encompassed within the spirit of the technology as defined by the scope of the claims. It will be readily apparent to one skilled in the art that varying substitutions and modifications can be made to the technology disclosed herein without departing from the scope and spirit of the technology.

The technology illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the technology as claimed. Thus, it will be understood that although the present technology has been specifically disclosed by various embodiments and optional features, modification and variation of the concepts herein disclosed can be resorted to by those skilled in the art, and that such modifications and variations are considered to be within the scope of this technology as defined by the appended claims and statements of the technology.

The technology has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

### REFERENCES

1. Lorane Texari, et. al. An optimized protocol for rapid, sensitive and robust on-bead ChIP-seq from primary cells. STAR Protocols 2, 100358, Mar. 19, 2021.
2. Orlando, D. A. et al. Quantitative ChIP-Seq Normalization Reveals Global Modulation of the Epigenome. Cell Reports 9, 1163-1170 (2014).
3. Bonhoure, N. et al. Quantifying ChIP-seq data: a spiking method providing an internal reference for sample-to-sample normalization. Genome Res 24, 1157-1168 (2014).
3. Shah et al. Examining the roles of H3K4 methylation states with systematically characterized antibodies. Mol Cell. 72, 162-177 (2018).
4. Guertin, M. J., Cullen, A. E., Markowetz, F. & Holding, A. N. Parallel factor ChIP provides essential internal control for quantitative differential ChIP-seq. Nucleic Acids Research 46, e75 (2018).
5. Egan, B. et al. An Alternative Approach to ChIP-Seq Normalization Enables Detection of Genome-Wide Changes in Histone H3 Lysine 27 Trimethylation upon EZH2 Inhibition. PLOS ONE 11, e0166438 (2016).
6. Vale-Silva, L. A., Markowitz, T. E. & Hochwagen, A. SNP-ChIP: a versatile and tag-free method to quantify changes in protein binding across the genome. BMC Genomics 20, 54 (2019).
7. Martin, B. J. E. et al. Transcription shapes genome-wide histone acetylation patterns. Nat Commun 12, 210 (2021).
8. Wang, Z. et al. Prediction of histone post-translational modification patterns based on nascent transcription data. Nat Genet 54, 295-305 (2022).
9. Okabe, A., Huang, K. K., Matsusaka, K., Fukuyo, M., Xing, M., Ong, X., . . . & Kaneda, A. (2020). Cross-species chromatin interactions drive transcriptional rewiring in Epstein-Barr virus-positive gastric adenocarcinoma. Nature Genetics, 52(9), 919-930.
10. Michael P Meers, Terri D Bryson, Jorja G Henikoff, Steven Henikoff (2019) Improved CUT&RUN chromatin profiling tools eLife 8:e46314.
11. Enrique Blanco, Luciano Di Croce, Sergi Aranda, SpikChIP: a novel computational methodology to compare multiple ChIP-seq using spike-in chromatin, NAR Genomics and Bioinformatics, Volume 3, Issue 3, September 2021, lqab064, https://doi.org/10.1093/nargab/lqab064
12. Zaijun Ma, Hui Wang, Yuping Cai, Han Wang, Kongyan Niu, Xiaofen Wu, Huanhuan Ma, Yun Yang, Wenhua Tong, Feng Liu, Zhandong Liu, Yaoyang Zhang, Rui Liu, Zheng-Jiang Zhu, Nan Liu (2018) Epigenetic drift of H3K27me3 in aging links glycolysis to healthy longevity in Drosophila eLife 7:e35368
13. Greulich, F., Wierer, M., Mechtidou, A., Garcia Gonzales, O., & Uhlenhaut, H. The Glucocorticoid Receptor Recruits the COMPASS Complex to Regulate Anti-Inflammatory Transcription. Available at SSRN 3658466.
14. Weiner, A., Lara-Astiaso, D., Krupalnik, V. et al. Co-ChIP enables genome-wide mapping of histone mark co-occurrence at single-molecule resolution. Nat Biotechnol 34, 953-961 (2016). https://doi.org/l0.1038/nbt.3652
15. Osipovich A B, Gangula R, Vianna P G, Magnuson M A. Setd5 is essential for mammalian development and the co-transcriptional regulation of histone acetylation. Development. 2016 Dec. 15; 143(24):4595-4607. doi: 10.1242/dev.141465. Epub 2016 Nov. 18. PMID: 27864380; PMCID: PMC5201031.

What is claimed:

1. A method for normalizing chromatin immunoprecipitation (ChIP) sequence data comprising:
obtaining a culture of animal cells;
crosslinking the animal cells;
harvesting the animal cells;
sonicating the animal cells to produced sheared chromatin;
spiking the sheared chromatin with at least two exogenous chromatin samples from the same species as the animal cells to produce a combined chromatin sample or two species that are different from the species of the animal cells to produce a combined chromatin sample, wherein the ratio of the amount of the at least two exogenous chromatin samples is known;
immunoprecipitating the combined chromatin sample;
producing a ChIP-seq library;
amplifying the ChIP-seq library;
sequencing the immunoprecipitated combined chromatin sample to produce sequencing data; and
normalizing the sequencing data based on the known linear relationship between the at least two exogenous chromatin samples.

2. The method of claim 1, wherein the animal cells are arrested in a prometaphase-like state.

3. The method of claim 1, wherein the animal cells are crosslinked in Dimethyl pimelimidate (DSG) with phosphate buffered saline (PBS), formaldehyde, or both.

4. The method of claim 1, wherein the ratio of the amount of the at least two exogenous chromatin samples is different.

5. The method of claim 1, wherein an antibody used in immunoprecipitating the chromatin recognizes an epitope in the chromatin and in the at least two exogenous chromatin samples.

6. The method of claim 1, wherein normalization is quantitative.

7. The method of claim 1, wherein a liquid handler is used in at least one of the steps of sequencing, immunoprecipitating, producing the ChIP-seq library, amplifying, or normalizing.

8. The method of claim 1, wherein the chromatin in the at least two exogenous chromatin samples are sheared.

9. The method of claim 1, wherein DNA in the at least two exogenous chromatin samples from the same species as the animal cells are barcoded while the DNA is still organized as chromatin.

10. A method of using an automated liquid handler to conduct ChIP-seq reactions comprising:
obtaining a culture of animal cells;
arresting a cell cycle of the animal cells;
crosslinking the animal cells;
harvesting the animal cells;
sonicating the animal cells to produced sheared chromatin;
input the sheared chromatin into a sample plate in an automated liquid handler;
actuate the automated liquid handler to at least one of:
immunoprecipitate the sheared chromatin;
wash and elute the immunoprecipitated sheared chromatin;
prepare a library;
amplify the library;
quantify DNA in the library;
sequence the library.

11. The method of claim 10, wherein a cell cycle of the animal cells is arrested before crosslinking the animal cells.
